(19) United States
(12) Patent Application Publication
(10) Pub. No.: US 2010/0120754 A1
(43) Pub. Date: May 13, 2010

(54) IDENTIFICATION AND USE OF SMALL MOLECULES TO MODULATE TRANSCRIPTION FACTOR FUNCTION AND TO TREAT TRANSCRIPTION FACTOR ASSOCIATED DISEASES

(76) Inventors: Peter Oettgen, Brookline, MA (US); Alan Rigby, Newton, MA (US); Towia Libermann, Newton, MA (US)

Correspondence Address: EDWARDS ANGELL PALMER & DODGE LLP, P.O. BOX 55874, BOSTON, MA, 02205, US

(21) Appl. No.: 12/436,685

(22) Filed: May 6, 2009

Related U.S. Application Data
(63) Continuation of application No. PCT/US2007/023429, filed on Nov. 6, 2007.
(60) Provisional application No. 60/857,407, filed on Nov. 6, 2006.

(51) Int. Cl. A61K 31/54; C40B 30/02; A61K 31/36; A61K 31/522; A61K 31/17; A61K 31/13; C07D 279/06; C07D 473/00; C07D 317/44; C07C 273/00; C07C 211/00

(52) U.S. Cl. 514/224.2; 506/8; 514/466; 514/263.37; 514/597; 514/645; 544/53; 544/276; 549/443; 564/49; 564/428

(57) ABSTRACT

The present invention relates to methods of identifying small molecule candidate agents capable of modulating transcription factor function such that the function/expression of a target transcription factor and/or proteins downstream of this target protein comprises the screening of small molecule libraries using in silico high throughput docking for candidate small molecules/agents that are selectively identified for their ability to target and disrupt the transcription factor-DNA interface through unique transcription factor and/or DNA descriptors that are defined within a pharmacophore, and then testing/evaluating the candidate agents identified above through one or more in vitro assays for their ability to modulate transcription factor function including expression of this target protein and/or proteins that are downstream of the target transcription factor.

The present application claims the benefit of U.S. provisional application No. 60/857,407 filed Nov. 6, 2006, which is incorporated herein by reference.

### STATEMENT OF GOVERNMENT SUPPORT

Funding for this invention was provided in part by the Government of the United States of America National Institutes of Health Grant PO1 HL76540. The Government has certain rights in this invention.

### FIELD OF THE INVENTION

The present invention relates to the identification and use of small molecules which modulate the interaction between transcription factors and DNA, and thereby affecting gene regulation and downstream protein expression or function.

### BACKGROUND OF THE INVENTION

In molecular biology, a transcription factor is a protein that regulates the activation of transcription in the eukaryotic nucleus. Transcription factors localise to regions of promoter and enhancer sequence elements either through direct binding to DNA or through binding other DNA-bound proteins. They act by promoting the formation of the preinitiation complex (PIC) that recruits and activates RNA polymerase.

Regulation of gene transcription by TFs is a central paradigm of eukaryotic biology for which Kanberg received the 2006 Nobel Prize. TF-DNA recognition is an intricate process involving spatial and temporal intermolecular contacts that are requisite for regulating downstream biological processes including; replication, DNA repair and initiation of transcription. These sequence specific TF-DNA interactions are mediated by a series of chronological, conformational perturbations by both the TF and DNA that facilitate molecular recognition and complex assembly on a dynamic time scale.

A global analysis of amino acid conservation within the TF-DNA interface has provided generalized “rules” for these interactions, however specific rules for this “class” of interaction interface are best understood in the context of intra-family comparisons (22-24). That said the structures of several ETS family members in the presence of promoter DNA sequences (ie. Ets-1, fli-1, GABPa, PDEF) have firmly established a structural paradigm of sequence specific ETS-DNA interactions involving both the core recognition sequence of the ETS binding sites (EBSs) and the 5′-/3′-flanking sequences (25-29). These and other studies have identified sequence diversity within the flanking regions of the DNA as well as the presence of unique amino acids localized within the DNA binding determinants of each ETS TF that permit each ETS DNA Binding Domain (DBD) to recognize unique EBSs and thus regulate the expression of multiple and unique classes of genes downstream of each, unique family members (21, 30).

The ETS family of transcription factors is comprised of 28 members that share a highly conserved DBD that is responsible for interacting with core EBSs containing the GGA(A/T) sequence. As previously mentioned the sequence flanking this core EBS also possesses critical descriptors that contribute to the specificity and selectivity of individual family members (18, 31-34). ETS TFs are autoregulated TF's involved in both the activation and repression of downstream target genes that are critical in a variety of disease pathologies including; inflammation, oncogenesis, apoptosis and angiogenesis (19, 20, 32, 35). It is well established that this DNA binding specificity is mediated by P—P interactions involving the ETS DBD and other domains within the ETS proteins including the pointed domain (21). This specificity is additionally regulated by the TF-DNA interface, which is comprised of both conserved and non-conserved amino acids arranged in a winged helix-turn-helix (w-HTH) structure (36) composed of 3 α-helicies (H1, H2, H3), 4 β-strands and “wing-like structures” that are believed to be responsible for critical contacts with the DNA minor groove (18, 29, 37, 38). Previous investigations have clearly established that several of the highly conserved residues including two invariant arginine residues (Arg391 and Arg394) positioned within the recognition helix, H3 that mediate bidentate contacts with the GG dinucleotides positioned within the core EBS are responsible for anchoring the ETS domain within the DNA major groove (2, 29, 30). However, high-resolution structures of several ETS family members have also identified mechanistic roles for other residues within the canonical DBD ((29) and references therein). Residues that mediate critical phosphate backbone contacts within the DNA minor groove are localized to the turns separating helices H2 and H3 and β-strands 3 and 4 ((14) and references therein). Numerous X-ray crystallography and Nuclear Magnetic Resonance (NMR) structural studies of ETS family members bound to high affinity DNA promoter sequences as well as, low affinity DNA promoter sequences both with and without adapter/repressor proteins have provided structural snapshots that clearly identify atomic details requisite to the sequence specific DNA binding and thus molecular mechanism of these TF-DNA interactions ((2, 21, 30, 39-41)} and references therein). Several X-ray crystal structures of Ets1 have elegantly defined subtle structural perturbations within the DBD resulting from its interaction with different promoter sequences (5′-GGAA/T-3′ versus 5′-GGAG-3′) both in the absence and presence of PAX5, a transcriptional regulator ((2) and references therein).

Although the expression of Ets-1 was originally believed to be restricted to lymphoid tissues in both T- and B-cells during their development (21) it is also expressed in endothelial cells (EC's) and vascular smooth muscle cells (VSMCs). The level of Ets-1 expression, which is upregulated in several invasive and metastatic solid tumors, is associated with the grade of malignancy and prognosis in several tumor types including breast, lung and colorectal cancer (16, 17, 21). Ets-1 has been shown to regulate genes involved in endothelial cell (EC) function, enhanced endothelial migration and angiogenesis (42, 43). In angiogenesis Ets-1 regulates the expression of other genes including the VEGF receptors, Flt-1 and Angiopoietin-2 (44-48). Although a role for Ets-1 in vascular development and angiogenesis in ECs has been shown, only recently did this group and others define a role for ETS TFs family, including Ets-1 in regulating vascular-specific gene expression. These findings identify Ets-1 as a critical mediator of vascular inflammation that is responsible for mediating inflammatory responses in a number of vascular diseases (49). Furthermore, these studies demonstrated that Ets-1 is a critical modulator of inflammatory responses in VSMCs in response to inflammatory stimuli that are upregulated in response to PDGF, Angiotensin II, and thrombin. Taken together, Ets-1 appears to be a promising target for selective therapeutic strategies since targeting this protein not only inhibits proliferation and resistance to apoptosis directly, but also inhibits tumor growth, invasion and metastasis indirectly through angiogenesis (17).

While there are several approaches to identify small molecule modulators of P—P and/or TF-DNA interactions, the two most adopted screening strategies are 1) experimental/biological HTS and 2) virtual screening or in silico high throughput docking (HTD), which is a computational approach that is driven by structural knowledge of the ligand and/or target being screened (73). The success of experimental HTS is limited by the inherent sensitivity of a user-constructed assay that is developed to evaluate a testable biological function of the target being explored. Cellular assays do not define the target actually affected. In addition to the time and resources needed for assay development these experimental HTS approaches suffer from reproducibility, cost and marginal success rate issues (74-76). While these issues

often represent a cost-benefit barrier for most academic researchers they are rationalized within the pharmaceutical industry due to the fact that many HTS assays and the chemical libraries that are screened by them are optimized for preferred “industry” targets such as G-protein coupled receptors (GPCRs), nuclear receptors, ion channels and enzyme targets (77). Although these targets have provided many novel therapies they represent only a fraction of the “druggable” subset of the human genome (>1,000 druggable genes), with many potential therapeutic targets under-exploited based on what appears to be a misperception that they are intractable to small, orally bioavailable molecules ((78-83) and references therein).

It would be useful to have effective methods of treating different types of diseases and disorders that are associated with transcription factors, in particular Ets-1. This specific transcription factor appears to be involved in diseases involving inflammation, including arthritis, inflammatory bowel disease and vascular inflammation. In addition, Ets-1 also appears to act as an angiogenic mediator in several types of cancer and optical diseases. Thus, it would also be useful to have methods of screening compounds that are capable of modulating the function and/or expression of the transcription factors involved in disease.

### SUMMARY OF THE INVENTION

The present invention relates to an alternative strategy for small molecule discovery by virtual screening or in silico high throughput docking (HTD), which is ideally suited for the rapid exploration of novel target space allowing uncharted biochemical and thus potentially therapeutic territory including that represented by the TF-DNA interaction interface to be explored in a cost effective manner (84-86).

Molecular recognition that is governed by TF-DNA interactions is the cornerstone of cellular function, mechanistic signal transduction and gene expression. These interfaces represent therapeutically interesting and commercially lucrative target space ((5, 6) and references therein). Although these interfaces were believed to be refractory to small molecule intervention, an improved understanding of these complex surfaces and the relative energetic contributions bestowed by; interface shape, interface size, geometrical complexity, polarity and roughness have recently stimulated renewed interest in this target space (6, 24, 63, 87, 88). Although TF-DNA interfaces are comprised of “interaction hot spots”, these important localized regions of interest differ in their compositional arrangement and type of determinants. The notion of “interaction hot spots”, which suggests that critical contact regions contribute a disproportionate amount of binding energy to the interaction, has provided a unique target strategy for the identification of small molecule “hits” for this chemical space which have been validated for P—P interactions (5, 23, 24, 63). These hot spots tend to be clustered within the interaction interface, thereby contributing in a manner to the formation of the complex through surface complementary, protein and DNA flexibility (89, 90).

Structure-based virtual screening or HTD of large publicly accessible chemical repositories into a well-defined TF-DNA interface that is critical for regulating aberrant gene transcription offers a unique strategy for the identification and development of transcriptional therapies (4, 6, 76). The methods of the present invention involve computational evaluation of publicly accessible chemical repositories using the HTD approach in an attempt to identify small molecule “hits” that are able to disrupt the Ets-1 MCP-1 promoter interaction (29). The National Cancer Institute (NCI) Diversity Set library and its parent NCI library of approximately 140,000 compounds has been screened in silico using our pharmacophore driven HTD approach (other libraries are also available), which has provided proof of concept data demonstrating that the interaction between Ets-1 and its cognate DNA sequence can be targeted and inhibited. Using the methods of the present invention, it has been demonstrated that several of the compounds inhibit the Ets-1/DNA interaction using electrophoretic gel mobility shift assays (ESMAs) and transactivation assays. These methods provide for the de novo identification of active compounds targeting the Ets-1/DNA interface. The HTD approach and in vitro validation data when combined with the structural studies as described herein provide a solid platform to identify and subsequently optimize new targeted transcriptional therapies.

In particular embodiments, the present invention relates to methods of identifying small molecule candidate agents capable of modulating transcription factor function such that the function/expression of a target transcription factor and/or proteins downstream of this target protein comprises the screening of small molecule libraries using in silico high throughput docking for candidate small molecules/agents that are selectively identified for their ability to target and disrupt the transcription factor-DNA interface through unique transcription factor and/or DNA descriptors that are defined within a pharmacophore, and then testing/evaluating the candidate agents identified above through one or more in vitro assays for their ability to modulate transcription factor function including expression of this target protein and/or proteins that are downstream of the target transcription factor.

Furthermore, the candidate agents comprised of unique chemical scaffolds as identified above are optimized for their ability to modulate the function and/or expression of the transcription factor target protein and/or proteins downstream of this transcription factor comprising testing the candidate agents in assays such as: 1) in silico quantitative structure activity relationships; 2) similarity fingerprint searching; and 3) NMR spectroscopy driven structural chemistry, wherein optimization results in greater modulation of the expression or function of target proteins that are downstream of the target transcription factor and/or improved pharmacokinetic, pharmacodynamic properties. The modulation can be represented by protein expression down-regulation or up-regulation.

In a preferred embodiment, the transcription factor is a member of the ETS family of transcription factors and, in particular, Ets-1.

In additional embodiments, the methods of identifying small molecule candidate agents capable of modulating transcription factor function can employ the initial selection of candidate agents selected from databases such as the Znc Database, National Cancer Institute's Diversity Set, the National Cancer Institute's Open Chemical Repository the Chembridge Library DIVERSet, the Maybridge Library, the Platinum Collection from Asinex and Natural Product Libraries.

The pharmacophore descriptors included for the transcription factor protein can comprise of hydrogen bond acceptors, hydrogen bond donors, hydrophobic disposition and the geometry of the protein's molecular scaffold (DBD backbone) and critical amino acids within the DNA binding domain of the transcription factor target. Also, the pharmacophore definitions for the protein can be computationally determined using a genetic algorithm and have been demonstrated to be critical through mutagenesis data, DNA binding data and/or other in vitro assays as identified within the supporting documentation.

Still other embodiments of the present invention relate to methods of preventing or treating a patient with a disease/disorder or susceptibility to a disease/disorder involving a transcription factor comprising the administration to the patient in need of such treatment or prevention a therapeutically effective amount of a compound identified by a methods described above. These diseases and/or disorders can involve inflammation such as rheumatoid arthritis, inflammatory bowel disease, atherosclerosis. bacterial sepsis and other diseases involving the immune system. In addition, the present invention includes treatment of diseases and/or disorders involving angiogenosis, such as various types of cancer (e.g., prostate, breast, colon, ovarian, lung and/or stomach cancers) and ocular diseases. The invention also includes treatment of cancer including e.g. prostate, breast, colon, ovarian, lung and/or stomach cancer.

The present invention also includes the use of compounds identified by the methods described for treating diseases and/or disorders as also described above. These compounds include compounds of Formula I and salts thereof. In particular, Compound 5b″:

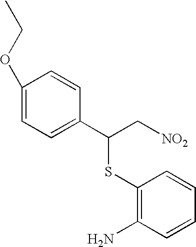

(also known as NCI 371776, 241-(4-Ethoxy-phenyl)-2-nitro-ethylsulfanyl]-phenylamine), and “Compound 28”:

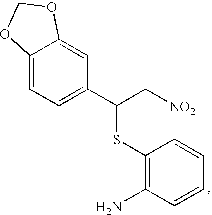

also known as NCI 371777, 2-{[1-(1,3-benzodioxol-5-yl)-2-nitroethyl]thio}aniline, can be used in the methods of treatment of the present invention. Additional compounds useful in the compositions and methods of the invention include:

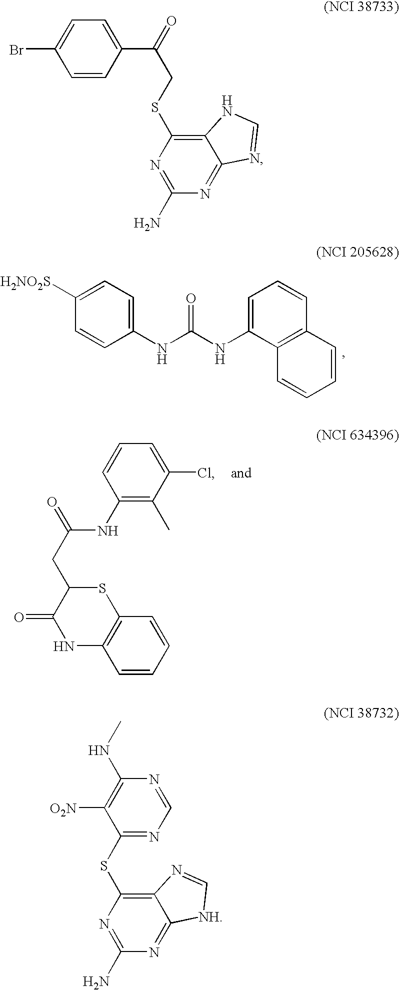

The pharmaceutically acceptable esters, salts, and prodrugs of these compounds are also contemplated for use in the compositions and methods of the invention. In certain embodiments, these compounds can be non-peptidic and have a molecular weight of less than 500.

Furthermore, the present invention includes the use of compounds represented by the formula A-D, in which A is an aromatic moiety or other molecular fragment capable of interacting with a DNA Binding Domain of a Transcription Factor involved in inflammation and or angiogenisis (and all related disease indications); and D is a moiety capable of interacting with a nucleic acid to which the transcription factor binds; wherein the compound is capable of modulating the ability of the transcription factor to bind to the nucleic acid; and pharmaceutically acceptable esters, salts, and prodrugs thereof, wherein the compounds are capable of modulating the function and/or expression of the transcription factor target and subsequent downstream proteins.

Another embodiment of the present invention includes pharmaceutical compositions comprising a compound identified by the methods of the present invention or a pharmaceutically acceptable salts thereof, together with a pharmaceutically acceptable carriers. Also included are pharmaceutical compositions comprising a compound of the invention (e.g., a compound listed hereinabove), or pharmaceutically acceptable salts thereof, together with a pharmaceutically acceptable carriers.

Further, the present invention includes kits for treating an diseases and/or disorders involving inflammation or angiogenisis in a subject. The kit is comprised of a identified by the methods of the present invention, pharmaceutically acceptable esters, salts, and prodrugs thereof, and instructions for use.

### BRIEF DESCRIPTION OF THE DRAWINGS

FIG. 1 is a schematic illustration of Ets-1 bound to its high affinity DNA sequences.

FIG. 2 presents schematic illustrations of using the X-ray structure of Ets-1 DBD in complex with its high affinity core sequence; ETS peptides were synthesized (A-E) and assessed their ability to block ETS/DNA gel mobility and transactivation assays.

FIG. 3 A illustrates that by using the crystal structure of the Ets-1 DBD in complex with DNA, the theoretical secondary structure and sequential position of peptides can be ascertained; B-C, illustrates the ability of the synthetic ETS peptides to compete with and/or block DNA binding in EMSAs and reporter gene transactivation assays.

FIG. 4 illustrates that the addition of the HIV-TAT protein-transducing domain to a peptide facilitated peptide transduction across the membrane of human primary endothelial cells and interestingly nuclear accumulation of the peptide in endothelial cells.

FIG. 5 illustrates A) Ets-1 DBD bound to promoter sequence, with separated surface illustrated in red, B) the P-D interface that we are targeting illustrated as a translucent Connolly surface and C) rotation of figure B (90° toward you) with a solid surface illustrating the hydrophobic interface (yellow), with compound #28 docked.

FIG. 6 illustrates that Compound 28 inhibits transactivation assays 8-fold.

FIG. 7 illustrates the expression and subsequent nickel column purification of the Ets-1 DBD fusion protein by SDS PAGE.

FIG. 8 illustrates that when human umbilical vein endothelial cells (HUVECs) were preincubated with 10 μM of our NCI “hit” compound (#28), it inhibits Ets-1 DNA binding.

FIG. 9 illustrates the overall steps involved in the methods of the present invention in identifying small molecules (candidate agents) which modulate the interaction between transcription factors and DNA.

FIG. 10 illustrates the structural domains of the Ets-1 protein which include the PNT, transactivation domain (TAD), ETS DNA binding domain and inhibitory domains (ID); reversible activity of the inhibitory domains is regulated through phosphorylation and protein-protein interactions.

FIG. 11 is a schematic of the structural domains of different ETS family members including the Ets domain (Ets); Pointed domain (PNT); Transactivation domain (TAD); Inhibitory domain (ID); A/T hook domain (A/T) and repressor domain (RD).

FIG. 12 illustrates the phylogenetic tree demonstrating the evolutionary relationship between ETS factor family members, based on the relative conservation of the Ets domain, linking members with closely homologous amino acid sequences; only human and drosophila ETS members are shown, with drosophila ETS family members (D-Ets-3, D-Ets-6, ELF, POINTED, D-Ets-4, E74 and YAN.

FIG. 13 illustrates the role of selected ETS family members (ESE-1), Ets-1, Elk-3) in the regulation of vascular inflammation in various cell types including endothelial cells (EC), vascular smooth muscle cells (VSMC) and mononuclear cells (MNC), in response to a variety of inflammatory stimuli.

FIG. 14 illustrates the role of selected ETS family members in innate and adaptive immunity with corresponding gene targets regulated by the particular ETS factors.

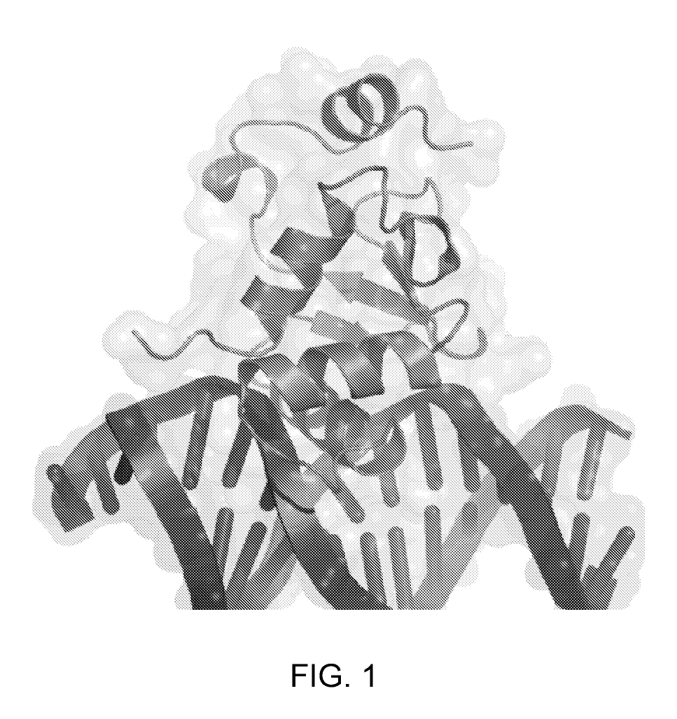

### DETAILED DESCRIPTION OF THE INVENTION

The present invention will now be described more fully hereinafter with reference to the accompanying figures, drawings or cited references by number in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs.

### I: DEFINITIONS

“Antisense RNA” refers to a single-stranded polynucleotide that is complementary to the mRNA produced from a gene. Antisense RNA hybridizes with and inactivates mRNA.

“Chemically synthesized,” as related to a sequence of DNA, means that the component nucleotides are assembled in vitro. Chemical synthesis of DNA may be accomplished using known procedures in the art. For example, automated chemical synthesis of DNA can be performed using one of a number of commercially available apparatus or vendors.

“Coding region” is the polynucleotide or that portion of a gene that codes for a specific RNA (sense or antisense) or polypeptide (i.e. a specific amino acid sequence), and excludes the 5′ sequence which drives the initiation of transcription. The coding region is typically the first polynucleotide(s) or the target polynucleotide(s) of the first nucleic acid and second nucleic acid, respectively.

“DNA” refers to deoxyribonucleic acid.

“DNA binding domain” or “DBD” refers to the region of a polynucleotide that encodes for the polypeptide portion of the transcription factor (TF) protein that enables the TF to bind to a DNA sequence.

“Downstream” refers to any element to the right of, or 3′ to, the coding region for a polynucleotide.

“Enhancer” is a DNA sequence which can stimulate promoter activity and may be an innate element of the promoter or a heterologous element inserted to enhance the level or specificity of a promoter.

“ETS-mediated Transcriptional Diseases/Disorders” refers to any condition involving TF-DNA interactions which result in or contribute to a disease or disorder including inflammation, angiogenisis, autoimmunity, arthritis, inflammatory bowel disease, cancer, artherosclerosis, bacterial sepsis, hypertension, restenosis, psoriasis and multiple sclerosis.

“ETS mediated Transcriptional Therapy” refers to sequence-specific TF-DNA interactions are spatially and temporally regulated, resulting in refined specificity/selectivity at the TF-DNA interface. TF-DNA interactions are critical mediators of gene expression that are regulated by extracellular signals that propagate information from the cell surface to the nucleus. The use of small molecule inhibits of the TF-DNA interaction interface provide a mechanism of transcriptional therapy through pathway specific transcriptional regulation.

“Expression” refers to the transcription of a gene or its polynucleotide region to yield sense RNA (i.e. mRNA) or antisense RNA encoded by the coding region. Expression also refers to the translation of mRNA into a polypeptide or protein.

“Gene” refers to a unit composed of a promoter region, a polynucleotide coding region and a transcription termination region, including any regulatory elements preceding or following the polynucleotide coding region.

“Heterologous” is used to indicate that a nucleic acid sequence (e.g., a gene) or a protein has a different natural origin or source with respect to its current host. Heterologous is also used to indicate that one or more of the domains present in a protein differ in their natural origin with respect to other domains present. In cases where a portion of a heterologous gene originates from a different organism the heterologous gene is also known as a chimera.

“Homologous” is used to indicate that a nucleic acid sequence (e.g. a gene) or a protein has a similar or the same natural origin or source with respect to its current host.

“Immune-related Diseases/Disorders” refer to health conditions for which the immune system is a component of the disease/disorder process, such as autoimmunity.

“Isolated” means altered “by the hand of man” from its natural state; i.e., that, if it occurs in nature, it has been changed or removed from its original environment, or both.

For example, a naturally occurring polynucleotide or a polypeptide naturally present in a living animal in its natural state is not “isolated,” but the same polynucleotide or polypeptide separated from the coexisting materials of its natural state is “isolated”, as the term is employed herein. For example, with respect to polynucleotides, the term isolated means that it is separated from the chromosome and cell in which it naturally occurs. As part of or following isolation, such polynucleotides can be joined to other polynucleotides, such as DNAs, for mutagenesis, to form fusion proteins, and for propagation or expression in a host, for instance. The isolated polynucleotides, alone or joined to other polynucleotides such as vectors, can be introduced into host cells, in culture or in whole organisms. Introduced into host cells in culture or in whole organisms, such DNAs still would be isolated, as the term is used herein, because they would not be in their naturally occurring form or environment. Similarly, the polynucleotides and polypeptides may occur in a composition, such as a media, formulations, solutions for introduction of polynucleotides or polypeptides, for example, into cells, compositions or solutions for chemical or enzymatic reactions, for instance, which are not naturally occurring compositions, and, therein remain isolated polynucleotides or polypeptides within the meaning of that term as it is employed herein.

“Messenger RNA,” also known as “mRNA” or “Sense RNA,” refers to a single stranded RNA molecule that specifies the amino acid sequence of one or more polypeptide chains.

“Minimal promoter” refers the minimal oligonucleotide or polynucleotide element necessary for transcription that contains a TATA-box.

“Nucleic acid” as used herein refers to DNA or RNA of genomic or synthetic origin which may be single- or double-stranded containing at least one gene that can encode for sense RNA or antisense RNA.

“Oligonucleotide” refers to a linear sequence of about 20 nucleotides or less joined by phosphodiester bonds.

“Operatively linked” generally refers to the association of various polynucleotide sequences of differing functions on a single nucleic acid or nucleic acid fragment so that the function of one polynucleotide sequence is affected by other sequence(s). In one example, with respect to the first polynucleotide(s), the first polynucleotide(s), the first promoter, the UAS1/UAS2, its optional terminator sequence and any optional regulatory elements are connected in such a way that the transcription of the first polynucleotide is controlled and regulated by the UAS1 and the first promoter. In another example, with respect to the target polynucleotide(s), the target polynucleotide(s), the second promoter and the UAS2, its optional terminator sequence and any optional regulatory elements are connected in such a way that the transcription of the target polynucleotide is controlled and regulated by the UAS2 and the second promoter. In another example, a promoter is operably linked with a coding sequence (i.e. the coding sequence is under the transcriptional control of the promoter). Coding sequences can be operably linked to regulatory sequences in sense or antisense orientation.

“Polynucleotide”, also known as a “DNA sequence”, refers to a linear sequence of about 20 or more nucleotides joined by phosphodiester bonds. In the polynucleotide DNA, the sugar is deoxyribose and in RNA, ribose. The polynucleotide may be single stranded or double stranded.

“Promoter” refers to the nucleotide sequences at the 5′ end of a gene or polynucleotide which direct the initiation of transcription. Generally, promoter sequences are necessary to drive the expression of a downstream gene. The promoter binds RNA polymerase and accessory proteins, forming a complex that initiates transcription of the downstream polynucleotide sequence. The promoter can include a minimal promoter that is a short DNA sequence comprised of a TATA-box and other sequences that serve to specify the site of transcription initiation, to which regulatory elements can be added for control of expression. The promoters for ETS factors do not contain a TATA-box. The promoter can also include a minimal promoter plus regulatory sequences that are capable of controlling the expression of a coding sequence or antisense RNA that is not translated. This type of promoter sequence consists of proximal and more distal upstream elements often referred to as “enhancers.” Promoters may be derived in their entirety from a native gene, or be composed of different elements derived from different promoters found in nature or even comprise synthetic DNA segments or oligonucleotides. It is understood by those skilled in the art that different promoters may direct the expression of a gene in different tissues or cell types, or at different stages of development, or in response to different environmental conditions (i.e. are inducible). Promoters which cause a gene to be expressed in most cell types at most times are referred to as constitutive promoters.

“Regulatory element” refers to polynucleotide(s) or DNA sequence(s) that play a role in determining promoter activity, i.e. a regulatory element can play a role in determining the activity of a regulatory sequence. Regulatory elements may affect the level, tissue/cell type specificity and/or developmental timing of expression. A regulatory element may be part of a promoter, or it may be located upstream or downstream of a minimal promoter. Polynucleotide sequences considered to be regulatory elements include sequences that have been shown to be target sites for binding of transcription factors, as well as sequences whose properties have not been defined but are known to have a function because their deletion from a promoter affects the expression.

“Restriction site” refers to a polynucleotide sequence at which a specific restriction endonuclease cleaves the plasmid, vector or DNA molecule.

“RNA” refers to ribonucleic acid.

“Small Molecule” refers to a compound with a molecular weight of up to approximately 1000 Da, including natural products and peptidomimetics.

“Target polynucleotide” refers to a polynucleotide which encodes for sense RNA (mRNA), antisense RNA, a polypeptide or a protein of interest.

“Terminator sequence” refers to a DNA sequence downstream of, or 3′ to, a coding sequence that causes RNA polymerase to stop transcription. The terminator sequence can include a polyadenylation sequence.

“Transgenic” is an adjective describing an organism (usually a plant or animal) that contains a transgene.

“Transgene” is a gene or DNA fragment that has been stably incorporated into the genome of an organism, such as a plant or an animal.

“Transcription” is the process by which a downstream nucleotide sequence is “read” to produce either messenger RNA (mRNA) or antisense RNA. The mRNA is the molecule that is “read” by the translational machinery to produce that protein. Variable regions at the beginning, i.e., 5′ end, and the end, i.e., 3′ end of the gene may or may not code for amino acids. Regions such as these are referred to as 5′ untranslated region (5′ UTR) and 3′ untranslated region (3′ UTR) respectively. A portion of the 5′ UTR serves as the binding region for the translational machinery (e.g., ribosomes and accessory proteins) required to synthesize a polypeptide encoded by an mRNA.

“Transcription Activation Domain” or “TAD” refers to the region of the ETS polypeptide sequence polynucleotide (i.e. the first or third polynucleotides) that encodes for the region of the transcription factor (TF) protein that facilitates activation of transcription when the TF is contacted with a complementary Upstream Activation Sequence (UAS1).

“Transcription factor” refers to a protein required for recognition by RNA polymerases of specific stimulatory sequences in eukaryotic genes. Such proteins activate transcription by RNA polymerase when bound to upstream promoters.

“Transformation” refers to any a process by which nucleic acids are inserted into a recipient cell to effect change. Transformation may rely on known methods for the insertion of foreign nucleic acid sequences into a eukaryotic host cell. Such

“transformed” cells include stably transformed cells in which the inserted DNA is capable of replication either as an autonomously replicating plasmid or as part of the host chromosome. They also include cells which transiently express the inserted DNA or RNA for limited periods of time.

“Upstream” refers to any element to the left of, or 5′ to, the coding region for a polynucleotide.

“Upstream Activation Sequence” or “UAS1” or “UAS2” refers to a nucleotide sequence (activation sequence) which can bind with a corresponding TF to activate transcription of a gene. The upstream activation sequence is located “upstream” or 5′ to the coding region for a polynucleotide.

In general, transcription factors are found to contain several functional domains, however one of theses is the DNA-binding domain and another one is for transcriptional activation. Transcription factors bind to and modulate the function of DNA. First, they bind specifically to their DNA-binding site, and secondly, they activate transcription. In addition, many transcription factors occur as homo- or heterodimers, held together by dimerization domains. For example, mutagenesis of the yeast transcription factors Gal4 and Gcn4 showed that their DNA-binding and transcription activation domains were in separate parts of the proteins.

### II: METHOD DESIGN FOR IN SILICO DISCOVERY OF SMALL MOLECULES

It is important to understand the mechanisms by which the critical mediators of transcriptional regulation (TF's) discriminate between promoter sequences within the genome. As previously noted this therapeutically important class of targets has been relatively intractable to small molecule inhibition and subsequent clinical intervention. Attempts to define the rules and/or principles governing these highly regulated interactions have permitted a better understanding of specificity, selectivity and conformational plasticity of TF-DNA recognition, which the methods of the present invention use to dock, score and rank small molecules in silico (22, 23, 62, 63). Structure-based virtual screening or high throughput docking (HTD) in which small molecules are docked into a target and scored on the basis of the energetic contributions of complimentarity, pocket shape, pocket size, geometrical complexity, polarity and roughness is an approach that has recently reemerged in preclinical development groups (111). This computationally powerful approach uses high resolution structural data of the target to guide the in silico virtual screening approaches (HTD), and provides an excellent opportunity for the discovery of small molecule “hits” that often require further in vitro characterization and/or optimization through QSAR or other approaches as previously defined (112, 113). Ets-1 was chosen for investigation due to its pivotal relationship in cancer and inflammatory diseases (17, 106).

### Pharmacophore-Guided Structure-Based HTD

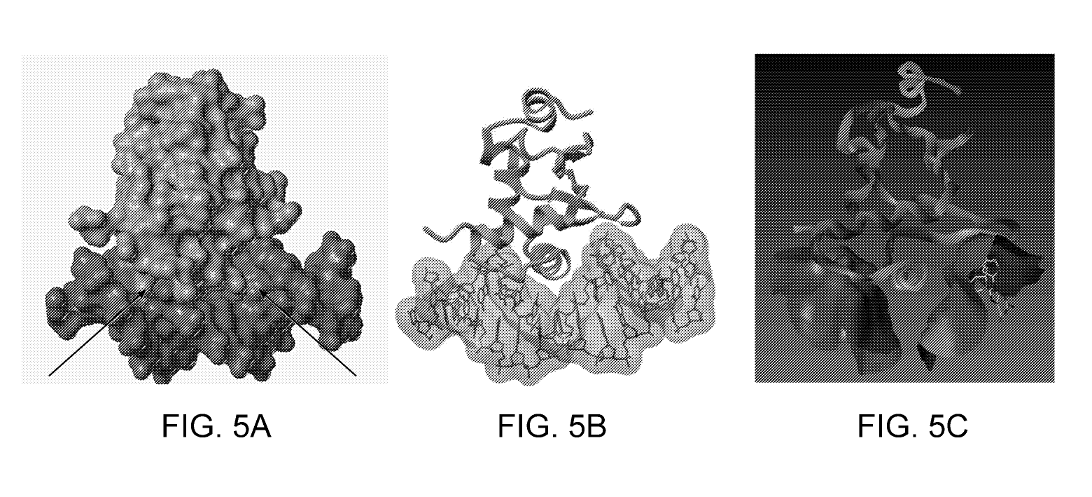

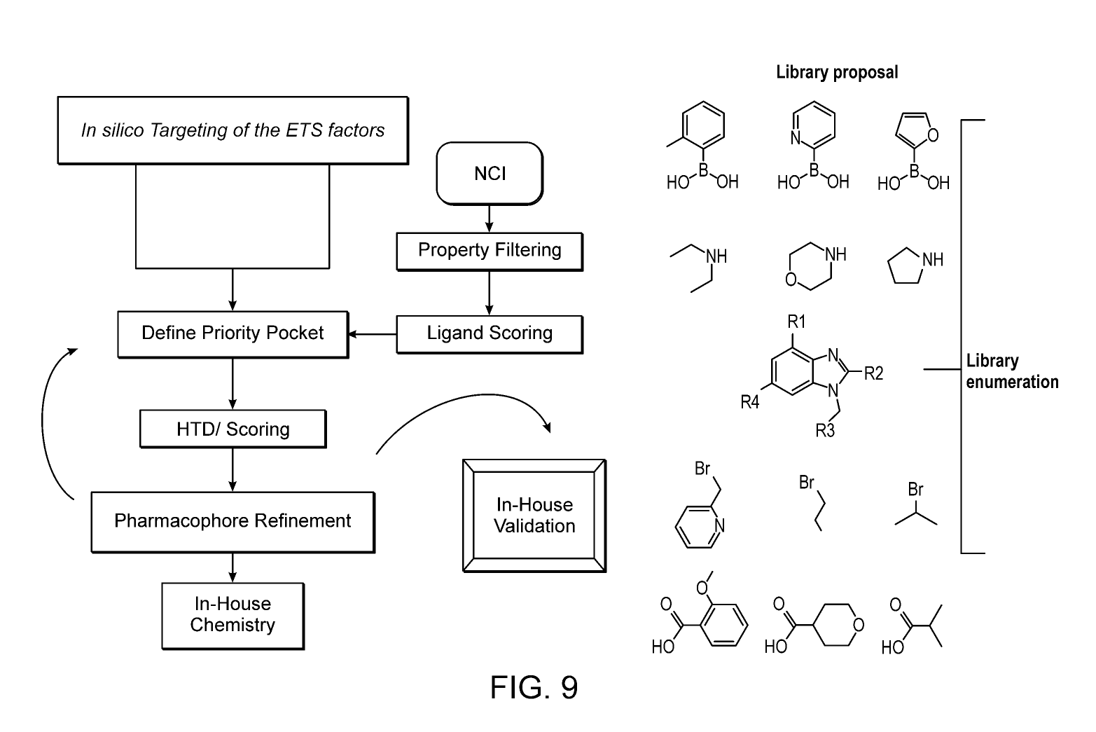

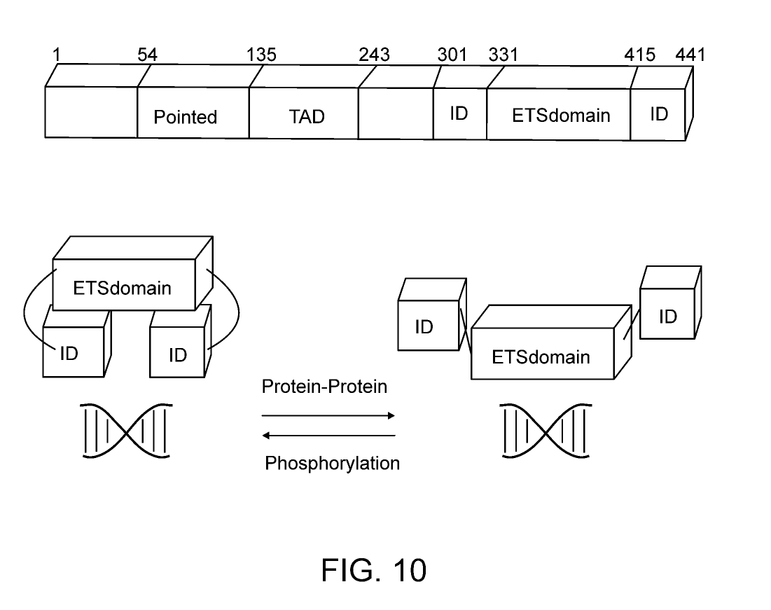

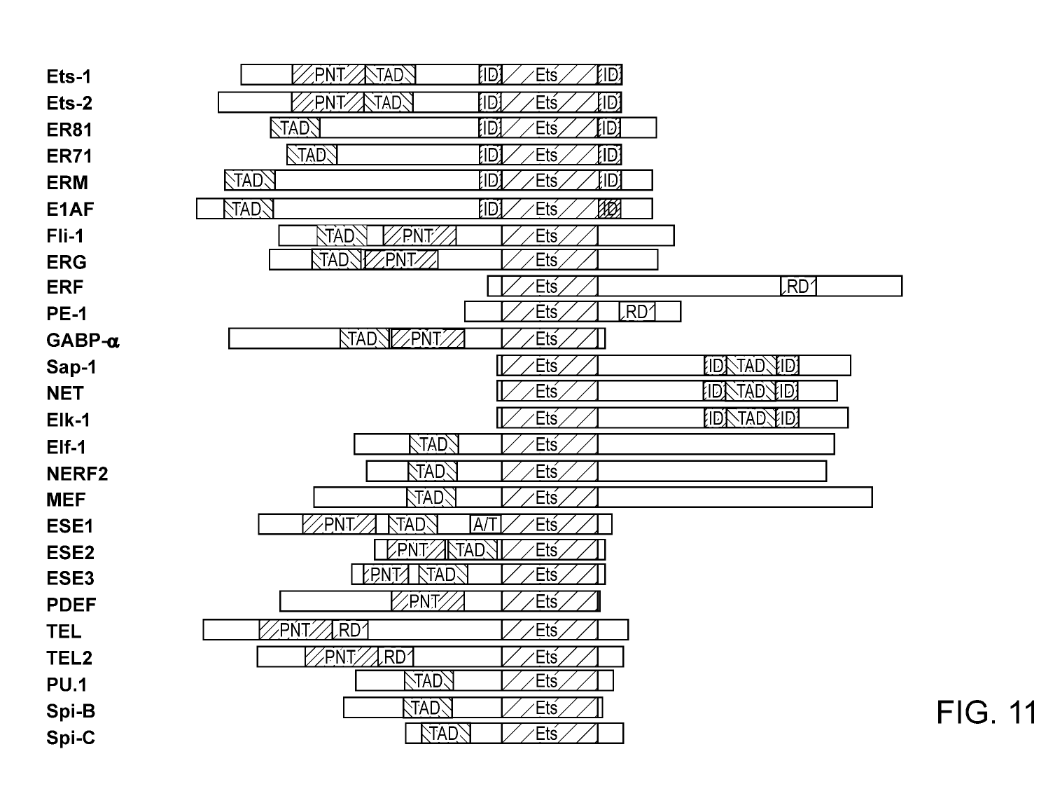

As the number of solved protein structures continues to increase through global structural proteomic initiatives, in silico, structure-based approaches for screening large compound repositories in search of small drug-like molecules targeted to these proteins becomes increasing important and is likely to provide novel therapeutics aimed at this new chemical space (i.e., druggable genome) (114). For HTD calculations of the present invention critical Ets-1 structural pharmacophores will be used that were [white arrows (FIG. 5)] identified by careful examination of the interaction interface between Ets-1 DBD and the DNA bases that flank the 5′ and 3′ ends of the core EBS sequence (illustrated in FIG. 9) and other requisite contacts identified from the crystal structures solved by Garvie and coworkers (2); (e.g., free ETS, ETS with inhibited modules, ETS and GGAG, ETS with PAX and GGAG, etc). These pharmacophores are comprised of a collection of discrete, critical physiochemical and structural attributes that are either directly connected through bonds (continuous) or are juxtaposed in 3D space by a molecular scaffold (discontinuous) (115, 116). Critical features of the pharmacophore such as hydrogen bond acceptors, hydrogen bond donors, and hydrophobic disposition are often easily identified by the superimposition of currently available ligands (the DNA) or can be computationally predicted using a genetic algorithm similarity program such as GASP (117-120). Another critical feature of successful pharmacophore design is the geometry of the molecular scaffold as it contributes to the structural rigidity and thus 3D presentation of the pharmacophore descriptors. The molecular scaffold geometry imparts additional conformational constraints upon the a-β bond vector (between the alpha carbon (Cα) and beta carbon (Cβ.) of the amino acid side chains that are critical for binding. In the case of Ets-1 this is particularly important for positioning Arg391, Arg394 and Tyr395, therefore we will define the atomic details of these residues with the EBS using extensive structural studies carried out on Ets-1 that have supported the importance of these residues when binding the EBS (2, 18, 25, 121). The inclusion of these pharmacophoric constraints results in faster triage of small molecules that cannot be reconstructed within the well defined, target binding site and facilitates a more conformationally expansive set of docking poses that more thoroughly sample the conformational space defined by these constraints.

The residue decisions are based on numerous structural studies of ETS family members in the absence of DNA, bound to high affinity DNA promoter sequences as well as low affinity DNA promoter sequences both with and without adapter/repressor proteins have provided structural snapshots that clearly identify atomic details requisite to the sequence specific DNA binding and thus molecular mechanisms of these TF-DNA interactions. This extensive, prior knowledge of the Ets-1DNA binding domain (DBD) the high resolution structure of which has been determined in the presence of its high affinity DNA sequence (5′-GGAA/T-3′), a lower affinity DNA binding sequence (5′-GGAG-3′) and in a ternary complex with DNA and the paired domain protein, Pax5 have been critical for performing HTD screens on Ets-1. Through the careful structural and functional analysis of these structures and others of ETS family members including the prostate specific ETS factor, PDEF critical pharmacophoric descriptors that are specific and thus selective for Ets-1, PDEF and ESE-1 have been defined. Although the ETS family members are highly homologous at the amino acid level several recent studies have demonstrated that specificity is mediated through limited degeneracy at the amino acid level within the TF-DNA interface of the Ets TFs. The atomic details afforded by the crystal structures of the Ets-1-DNA interface and recent elegant NMR experiments provide a mechanistic foundation for sequence specific DNA interactions. These pharmacophoric “hot spots” are used as structure-guided descriptors for structure-based HTD, in silico screens that are designed to identify small molecules that selectively target the Ets-1-DNA interface.

### High Throughput Docking, Using FlexX

The HTD approach of the present invention uses the SYBYL suite of programs including FlexX (TRIPOS, St Louis, Minn.), which considers the conformational flexibility within the target interface in combination with a powerful incremental construction algorithm that allows the complexity and size of the ligand to be iteratively constructed from a predefined base fragment (122). The algorithm used by FlexX is based on the model of molecular interactions defined by Bohm (123) and Klebe (124) and is divided into three segments: core or base selection, core placement, and incremental complex construction (125). Briefly, the small molecule to be docked (from the chemical library being screened) is initially fragmented resulting in the generation of several base fragments. Subsequent base fragment selection is highly dependent upon the number and specificity of contacts made between the respective fragments and the target active site and provides the algorithm with a single, preferred binding orientation. Two unique algorithms focused on resolving geometric ambiguity are responsible for placing the base fragment. FlexX differs from DOCK and other HTD programs in that the placement of the core fragment is based upon interaction geometries between the ligand fragments and target active site descriptors. These interacting groups and/or descriptors that define critical features of the active site are primarily hydrogen-bond donors and acceptors, as well as hydrophobic groups. Following base placement, the remaining constituents of the ligand are divided into small fragments and incrementally “grown” onto the base alternatives. This incremental construction method provides a tree search, with unproductive (energetically unfavourable) branches being pruned as quickly as possible within this iterative stage. Following the addition of incremental pieces of the ligand, the ligand is then ranked and the best ranked solutions or poses are kept at each level of the tree's growth ensuring that the energy of the ligand is minimized, while pose clustering removes similar configurations. Once the compound is docked a variety of scoring functions are available to assess the energy of each calculated pose, which takes into account the forces involved in this interaction and the affinity between the interacting compounds. This in silico, “virtual” approach to library selection is comprised of three main steps; virtual filtering, virtual profiling and virtual screening (112, 113, 126). The application of virtual filtering takes into account preferred ADME (adsorption, distribution, metabolism, expression) properties as defined by the Lipinski “rule of five” pharmacological guidelines that include molecular mass, lipophilicity, hydrogen bond donors/acceptors (hydrophilicity) (108). Although there are only four parameters that define this term, the name is coined based on the cutoff values for each of these parameters that are used in defining the “druglikeness” of the potential “hit” candidates being approximately 5. The virtual filtering and profiling steps are important for “qualifying” specific candidates. The virtual screening filters specific candidates using our pharmacophoric constraints that are unique to the TF-DNA interaction being studied. FlexX-Pharm, which is an extension of the flexible docking program FlexX that permits the inclusion of user defined pharmacophoric constraints that can be used in the methods of the present invention.

### Molecular Libraries to be Screened

The data presented herein is derived from our screens of the NCI Diversity Set, which is a compound collection representing a universally diverse group of “drug-like” small molecules chosen on the basis of their 3D pharmacophoric scaffolds, which represent diverse, biologically relevant pharmacophoric scaffolds from within the NCI parent library. This 1900 compound library was selected from a parental 140,000 compound library that has been created within the Developmental Therapeutics Program at the NCI http://dtp.nci.nih.gov/index.html. In addition, to screening the NCI libraries we will also continue to screen the Chembridge library (http://chembridge.com/chembridge/), a library of greater than 450,000 (and increasing) handcrafted small molecule compounds. This library has been selected from their master database of (>5 million compounds) ensuring computational diversity of the discrete chemical moieties, drug-like properties, as well as medicinal chemistry pharmacokinetics. One important advantage is that compounds that match our pharmacophoric query are available for purchase, are pure and have been subjected to NMR and mass spectrometry analysis to validate chemical composition and purity. Our preliminary Chembridge screens identified 138 compounds from a compound collection of 50,080. The Maybridge library is another alternative collection that is comprised of 60,000 organic compounds, produced by innovative synthetic techniques, representing ˜400,000 pharmacophores identified within the world drug index ˜87% (*calculations carried out by Oxford Molecular using Chem-X definition, i.e. triplets of H-bond acceptors, H-bond donors, aromatic ring centers and positive nitrogen atoms). Alternative compound libraries are available and a very recent compilation of one million commercially accessible compounds, including a natural product library, was made available for web-accessible database searching and docking through ZINC (http://blaster.docking.org/zinc) (111).

### Fingerprint Similarity Search for Additional Small Molecules Using Sub Structural Similarity Keys of “Hits”

The use of molecular fingerprints for similarity searching is a widely accepted approach to identify novel active compounds based on their similarity to one or more established, functional small molecule scaffolds (131). Briefly, this substructure searching compares a bit string representation of the “hit” structure with other small molecules within a database in search of overlap using various metrics. Although the Tanimoto coefficient is the most popular similarity metric, the inclusion of 2D and 3D structural and molecular descriptors are important (132). The median partitioning (MP) mini-fingerprint (MFP), MP-MFP similarity search encodes 61 property descriptors with 110 structural fragment-type descriptors and transforms these property descriptors into binary easy to translate descriptors will be used for our similarity searches due to its low false positive rate (0.04%), its effectiveness in several benchmark calculations and its accessibility (it is publicly available) (133-135). The present invention employs similarity searches for compounds that have structural or descriptor overlap (Tanimoto coefficient >0.65-0.85) with those NCI “hits” that are functionally validated in our in vitro studies. Initially the fingerprints of these “hits” are used to perform a MP-MFP similarity search with the parental NCI 140,000 compound library from which the Diversity Set of compounds originates (131, 135-138). The ability of MP-MFP to correlate bit string similarity to biological activity, while distinguishing inactive compounds has been recently validated for several biological targets (131). This approach can be used to screen the zinc repositories.

### Additional Approaches

Structure-based and ligand-based HTD is dependent on the docking and subsequent scoring algorithm used to; 1) accurately predict the correct pose of the active/“hit” and 2) reproducibly rank or score this active for correctness or tightness of fit, while simultaneously optimizing this computationally process for accuracy and speed (139, 140). One caveat of this in silico approach is that the pose that is predicted in silico to be the best and thus representative of the active conformation (as defined by crystallographic binding modes), is not always scored/ranked highest, which introduces the potential of false positives, further increasing the complexity of this approach. Inherent limitations in the scoring algorithms used to reproducibly rank identified “hits” within structure-based and/or ligand based small molecule discovery initiatives have impeded industry wide adoption of this approach alone for discovery initiatives, although virtual HTD is often used as a complementary approach to experimental HTS and/or partnered with structural approaches as described herein ((73) and references therein). If these structure-based virtual screening approaches are used alone these inherent caveats can be insurmountable. However many of these limitations can be overcome through an exhaustive iterative approach that partners in silico HTD with an approach for structurally validating these “hits” such as a detailed NMR analysis, that would permit “hits” identified by HTD that are false positives to be triaged much earlier in the discovery process (141).

### Nuclear Magnetic Resonance (NMR) Spectroscopy and Small Molecule Discovery

NMR is an established method for the three-dimensional structure determination of small proteins (=40 kDa) and is an archetypical method for characterizing the molecular dynamics that are critical for macromolecular complex assembly ((91, 92) and references therein). In recent years the molecular mass range amenable to structure determination by NMR has increased significantly (>50 kDa) with the development of triple resonance pulse sequence technologies, increased magnetic field strengths and heteronuclear recombinant protein expression methodologies (91, 93-95). Importantly, the ability of NMR spectroscopy to provide structural details for P—P and P-ligand interaction interfaces in protein complexes well beyond 100 kDa is invaluable for translating these binding data into interaction interfaces that are now, as previously noted, viable targets in an emerging paradigm of discovery initiatives in search of novel small molecule therapeutics (96). NMR spectroscopy provides a robust platform to characterize both the ligand binding site and affinity, while simultaneously providing a window through which the entire target protein(s) can be structurally observed without the need of an assay to detect this interaction. Successful small molecule screening and subsequent “hit” validation requires a biophysical approach that is capable of detecting relatively weak interactions such as those observed by NMR ((96-98) and references therein). The increasing use of NMR spectroscopy in drug discovery pipelines is due to the ability of NMR to detect ligand binding over many affinity ranges, while as noted providing detailed structural information for the entire target, which permits the identification of the specific ligand binding site ((99, 100) and references therein). Furthermore, NMR-based HTS, fragment screening, SAR by NMR, and other NMR applications involved in lead validation through optimization have been integrated into many discovery pipelines to facilitate earlier stage “false positive” triage ((95) and references therein). Demonstrable success with these NMR-based approaches supports an increasingly important role for NMR in both ligand and target validation, which will become increasingly significant as we extend the boundaries of conventional target space (101). The methods of the present invention involve extensive use of NMR knowledge to monitor the small molecule-binding site within Ets-1 and subsequently use these data for structure-guided validation and lead optimization.

### III: IN VITRO CHARACTERIZATION OF SMALL MOLECULE “HITS” THAT INHIBIT THE ETS-1 DBD RATIONALE

The computational analysis using HTD and/or structural similarity searches discussed above has lead to the identification of several candidate “hits” or small molecules that target the interaction between Ets-1 and the EBS in regulatory elements of downstream target genes. As illustrated in the data presented within several small molecules have been identified from the NCI Diversity Set of compounds that inhibit Ets-1-DNA binding and subsequent transactivation. These compounds represent several unique chemical scaffolds comprised of; a nucleoside analog containing the 2-amino-purine scaffold, the benzodioxl scaffold and an aniline system with a chiral center. Having identified small molecule “hits”, the ability of these compounds to abrogate the function of Ets-1 in vitro is evaluated. In doing so, several complimentary assays will be used including gel mobility shift assays, transactivation assays, and chromatin immunoprecipitation (ChIP) assays to identify the specificity and inhibitory concentration at which these small molecules are active. Compounds that specifically inhibit DNA binding of Ets-1, but not other Ets factors in these assays will be further evaluated in vitro to evaluate Ets-1 dependent gene regulation.

### In Vitro Evaluation of HTD Lead Compounds

### A) Electrophoretic Mobility Shift Assay.

Initially it is determined whether the identified small molecule “hits” interfere with Ets-1-DNA binding using electrophoretic mobility shift assays (ESMAs). These are performed as described previously (142). In brief, in vitro translated Ets-1 protein is generated using a rabbit reticulolysate system (Promega) and a mammalian expression plasmid encoding the Ets-1 protein. 1 μl of the in vitro translation reaction and 0.1-0.2 ng [32]dATP-labeled double-stranded oligonucleotide probe (5,000-10,000 cpm) will be run on 4% polyacrylamide gels containing 0.5× TBE buffer. An Ets-1 antibody (SantaCruz) will be used to demonstrate the specificity of the band. The small molecules identified are initially evaluated at a concentration of 1 mM, which is the concentration used in high throughput fraganomic approaches for evaluating the interaction of weak-binding small molecules (143, 144). Those small molecules that inhibit the Ets-1-DNA interaction will be re-evaluated over a concentration range of 10 μM-1 mM. Solutions containing DMSO alone will be used as a vehicle control. To ensure the small molecule is specifically targeting the Ets-1-DNA interface and not interacting non-specifically with the DNA, a non-specific promoter is included as well as a control small molecule that is not selected as a “hit” by our HTD screen. To further confirm the molecular specificity of these small molecules for targeting Ets-1, EMSAs will be performed with other members of the ETS family. Ethidium bromide displacement assays could also be used to confirm that the small molecules are not nonspecifically targeting the DNA sequence (145). The most potent small molecules identified will be evaluated further for their ability to inhibit the transactivation of several promoters by Ets-1.

### B) Mammalian Expression Vectors and Luciferase Reporter Gene Constructs.

Several downstream targets of Ets-1 including the MCP-1 gene, PAI1, and Flt-1 (49, 142) have been identified. To further define the specificity of small molecules that inhibit DNA binding of Ets-1 in EMSAs, their ability is evaluated with regard to inhibiting transactivation of the MCP-1, PAI-1, and Flt-1 promoters by Ets-1, without affecting the basal activity of the promoter. As controls, the endothelial-specific promoters Tie1 and Tie2 are used, that have been previously shown as targets of the Ets factors NERF2 and Elf-1, but not Ets-1 or Ets-2 (142, 146). The promoters of these genes have previously been subcloned into the PGL2 luciferase reporter (Promega). Similarly, we have previously subcloned the

cDNAs encoding the ETS factors Ets-1, Ets-2, NERF2, and Elf-1 into the PCI mammalian expression plasmid.

### C) DNA Transient Transfection Assays.

Human embryonic kidney cells (HEK 293) are cultured as previously described (147). HEK 293 cells are kidney epithelial cells that express low basal levels of Ets-1, and are easily transfected. Cotransfections of 2×105 HEK 293 cells are carried out with 0.3 μg of the reporter gene construct DNA and 0.15 μg of the mammalian expression vector encoding the selected Ets factors using 4 μl LipofectAMINE (Invitrogen, San Diego, Calif.) as described (148). Small molecules that inhibit DNA binding are tested, for their ability to inhibit transactivation of several different promoters by Ets-1. Those small molecules that also inhibit Ets-1 transactivation is further investigated for specificity by evaluating their ability to inhibit the transactivation of the Tie1 and Tie2 promoters, two promoters that are transactivated by other Ets transcription factors, NERF2 and Elf-1 (1). All experiments are performed in triplicate.

D) Chromatin immunoprecipitation (ChIP).

Chromatin Immunoprecipitation (ChIP) is performed as previously described to determine if Ets-1 interacts with specific EBS within the MCP-1, PAI-1 and Flt-1 promoters (49, 149, 150). Briefly, the TF-DNA complex in 2×106 primary HEK293 cells cotransfected with the mammalian expression plasmid encoding Ets-1. The transfection is carried in the presence of the small molecules or vehicle (DMSO at the same concentration that is used to dissolve the compounds). DNA-protein complexes are crosslinked by 1% formdeldhyde for 10 min in the culture medium and the reaction is stopped by the addition of 0.1M glycine. The cells are collected by centrifugation and washed in cold PBS plus 0.5 mM phenylmethylsulfonyl fluoride. Cells are collected by centrifugation and washed as above and then swelled/lysed in 5 mM pipes (pH 8.0), 85 mM KCl, 0.5% NP-40, 0.5 mM phenylmethylsulfonyl fluoride, and 100 ng/ml leupeptin and aprotinin, by incubation on ice for 20 min. Nuclei are collected by microcentrifugation at 5,000 rpm, resuspended in sonication buffer [1% SDS, 10 mM EDTA, 50 mM Tris.HCl (pH 8.1), 0.5 mM phenylmethylsulfonyl fluoride, and 100 ng/ml leupeptin and aprotinin] and incubated on ice for 10 min. The DNA is then sheared by sonication on ice to an average length of 500-1,000 by and then microfuged at 14,000 rpm. Immunoprecipitation, washing, and elution of the immunoprecipitated complexes are carried out as described using an Ets-1 polyclonal antibody (SantaCruz) (151). Before the first wash, the supernatant from the reaction lacking primary antibody for each time point is saved as total input of chromatin and is processed with the eluted immunoprecipitates beginning at the crosslink reversal step. DNA will be isolated by immunoprecipitation and analyzed by PCR using primers flanking specific Ets-1 binding sites within the MCP1, PAI-1, or Flt-1 promoters.

### Validation of the Specificity of the Compounds for Ets-1 Target Genes

In order to further define and validate the specificity of the HTD identified small molecules that target Ets-1, the transcriptional profile of the RNAs isolated in the experiment above is determined. 2 μg of total RNA from duplicate experiments as detailed (Preliminary data, C. 1.4) is used to generate the probes and hybridized to the Affymetrix U133 Plus 2.0 GeneChip that contains the whole human genome (>56,000 transcripts). An in-depth bioinformatic analysis will identify the set of genes that are; 1) induced or repressed by the pro-inflammatory cytokines in HUVEC cells at the different time points, 2) the genes that are induced or repressed by the small molecules in the absence of pro-inflammatory cytokines and 3) the genes whose induction or repression by pro-inflammatory cytokines is prevented by these compounds. Data is compared to data that identities the set of genes affected by the Ets-1 siRNA in order to determine the specificity of the different compounds for Ets-1 {Jung, 2005 #7491. The compounds that indeed inhibit Ets-1 DNA binding will also affect many genes that are affected by the Ets-1 specific siRNA. However, some of these compounds will target additional pathways that would be reflected by the changes in gene expression of genes not affected by the Ets-1 siRNA. Pathway modeling using Ingenuity's Pathway software enables greater precision in identifying biological pathways targeted by the different compounds. Briefly, the collection of genes that are regulated by the HTD compounds are further analyzed in the context of complex biological pathways. Iobion's PathwayAssist {Nikitin, 2003 #7511 and Ingenuity's Pathways Analysis (www.ingenuity.com) software will be used, both of which integrate proteins into biological pathways based on scientific literature by using natural language processors and expert human curation and have been used as successful tools for further hypothesis generation {Panda, 2002 #750}.

### Real Time PCR

SYBR Green I-based real-time PCR is carried out on an Opticon Monitor (MJ Research, Inc, Waltham, Mass.). All PCR mixtures contain PCR buffer [final concentration: 10 mM Tris-HCl (pH9.0), 50 mM KCl, 2 mM MgCl2 and 0.1% TritonX-100], 250 μM deoxy-NTP (Roche), 0.5 μM of each PCR primer, 0.5×SYBR Green I, 5% DMSO, and 1U Taq DNA polymerase (Promega, Madison, Wis.) with 2 μl cDNA in a 25 μl final volume of reaction mix. Then, the fluorescence signal is measured immediately following incubation at 78 oC for 5 s that follows each extension step, which eliminates possible primer dimer detection. At the end of PCR cycles, a melting curve will be generated to identify specificity of the PCR product. For each run, serial dilutions of human GAPDH plasmids are used as standards for quantitative measurement of the amount of amplified cDNA. For normalization of each sample, GAPDH primers are used to measure the GAPDH cDNA levels.

Cytotoxicity Evaluation of “hit” Compounds using MTS Assay

Cells are plated in 50 μL of growth medium in a 96-well plate format. After adhesion, the cells are treated with an additional 50 μL growth medium containing compound or DMSO only. Each analysis is performed in triplicate. Following compound treatment (22 hours with HEK 293 cells and 6 hours with HUVECs), the media is replaced and 20 uL CellTiter 96 AQueous One Solution Reagent (Promega), which contains a MTS tetrazolium compound that is soluble in tissue culture and reduced in cells into a colored formazan product is added to each well. The absorbance at 490 nm will be recorded using a 96-well plate reader immediately and every thirty minutes for two hours. Comparing the change in absorbance between treated cells and untreated control cells over the same time period will allow evaluation of the cytotoxicity of the HTD identified “hits”.

### Bioinformatics Analysis of Microarray Data

Sophisticated bioinformatics is necessary to interpret these expression data, which can be carried out by a bioinformatics specialist. The microarray data is analyzed in various aspects. First, the online annotation tool (www.bidmcgenomics.org) developed at the BDIMC Genomics Center, which combines more than 80 categorical information sources on any given gene, is used to further investigate results in relation to existing biological databases. This annotation software is a web-based interface that allows investigators to query a database providing information on gene accession numbers and to convert gene accession numbers into meaningful values. This annotation tool enables import of the whole data set of genes identified by transcriptional profiling and provides meaningful values including annotations from multiple databases such as the Gene Ontology annotations of the NCBI, biological function, cellular location, molecular function, biological pathways, disease relationship as well as whole summaries describing various aspects about the gene. This annotation tool rapidly determines the biological significance of the correlations, and clusters computed from the microarray measurements.

### Alternative Approaches

The results of the proposed experiments are used to determine if the small molecules identified by the methods described above are able to interfere with the binding of Ets-1 to DNA binding sites within the regulatory elements of known target genes including MCP-1, PAI-1, and Flt-1. However, it is possible that these small molecules may function only at higher concentrations, or lack Ets-1 specificity, thus necessitating optimization of these current “hits” and/or a rigorous structural similarity search to identify additional compounds that have scaffolds similar to the current “hits” or alternative parental scaffolds. Simultaneously, refinement of HTD pharmacophoric descriptors is done for further virtual screens of the NCI and other proprietary libraries. Small molecules such as compound #28 that demonstrate in vitro function for Ets-1 is further evaluated using NMR spectroscopy to characterize their mechanism of action, while providing the atomic details requisite for structure-guided optimization. Interestingly, HTD calculations have been performed on two additional ETS factors, including prostate derived ETS factor (PDEF) for which we used the recently determined 3D structure of PDEF bound to the PSA promoter (29) and Ese-1, which required that a homology model of the ESE-1 DBD to be generated using homology modeling (152). These studies identified a “hit” list for each of these ETS targets that is comprised of ˜30 unique small molecules of which only 2 are similar to those identified for Ets-1.

### IV: STRUCTURAL VALIDATION AND OPTIMIZATION OF IDENTIFIED HITS

TF-DNA recognition is an intricate process involving the formation of specific spatial and temporal intermolecular contacts that result in conformational perturbations in both the cognate DNA sequence and the TF. As noted the intricacies of the TF-DNA interface have been used previously to develop compounds such as intercalating agents, minor groove binders and triple helix oligonucleotides in an attempt to inhibit these critical interactions and thus regulate transcriptional activation (70, 71). In the present invention use NMR spectroscopy is used, which is an excellent approach for characterizing protein-ligand complexes (ie. Ets-1-Compound #28) over a large affinity range (96). NMR resonance frequencies for individual nuclei represent what is known as the ‘fingerprint’ of the protein structure. Ligand-induced changes in the chemical environment of nuclei localized at and within the binding site will conformationally perturb these resonances (chemical shifts), providing us with a highly sensitive tool for identifying the amino acids in Ets-1 that mediate this interaction. Each amino acid resonance within the Ets-1 DBD has been previously assigned and then use these assignments as a guide to monitor the site-specific conformational perturbation of residues involved in mediating interactions with the small molecule “hits” (25, 33, 121, 153). Should it be necessary the NMR structure of the co-complex will be determined using a strategies we have used extensively for protein structure determinations.

### A) Cloning, Expression, Purification and 2H/15N/13C-Labeling of the Recombinant Ets-1 DBD.

Our original gene insert for the human Ets-1 DBD, residues Ile335-Ser420, was prepared by PCR amplification of the appropriate region from full-length human Ets-1 cDNA using primers designed to facilitate directional cloning into the pET-32b expression vector (Novagen, Inc.). The Ets-1 DBD sequence was inserted downstream of a thioredoxin fusion sequence comprised, a six-residue histidine affinity tag and an enterokinase (EK) recognition site in the pET-32b vector. Although a strategy was developed for cleavage of the fusion protein and subsequent purification of this Ets-1 DBD that is expressed in the inclusion bodies, the additional steps necessary for obtaining this protein, which included solubilization prior to purification, and then renaturation using a stepwise reduction of urea by extensive dialysis may prove problematic when labeling this protein with 15N and/or 15N/13C for extensive structural studies. To circumvent many of these issues an additional construct is made as detailed by Garvie and coworkers that is expressed in the supernatant and requires no intermediary refolding steps (2). For structure-guided optimization of the “hit” compounds and/or co-complex structure determination we recombinantly express this Ets-1 DBD construct in M9 minimal media that contains 15N-ammonium chloride and/or 13C-glucose as the sole nitrogen and carbon source respectively. Briefly, this gene construct comprised of residues Gly331-Asp440 of the Ets-1 DBD, will be PCR amplified from human Ets-1 cDNA using primers: 5′-gcctcgacgccatgggcggcagtggaccaatc-3′ and 5′ cgggacctcggatccctagtcggcatctggctt-3′ designed to facilitate directional cloning into the pET-19b expression vector (Novagen, Inc.). Then this new construct is overexpressed in E. coli BLR(DE3) cells cultured in M9 minimal media supplemented with carbenicilin (50 μg/mL) at 37° C. until an optical density (A600 nm) of 0.5 is reached. Isopropyl b-D-thiogalacopyranoside (IPTG) will then added (1 mM) to induce expression from the bacteriophage T7/lac promoter for 4-6 hours (growth is typically slower in this minimal media). Cells from these induced cultures are harvested by centrifugation and resuspended in 500 mM NaCl, 5 mM DTT, 0.1% IGEPAL, 1 mM EDTA, 100 mM Tris (pH 8.0) and subsequently lysed via a microfluidizer. The filtered lysate is extensively dialyzed into 100 mM KCl, 5 mM DTT, 20 mM citrate buffer (pH 5.3) prior to ion exchange and size exclusion FPLC purification as has been detailed previously (2, 154). The Ets-1 DBD protein is concentrated to ˜0.5-1 mM using a centrifugal concentrator device (Amicon Ultrafree, Millipore).

### B) Investigate the Interaction Between Ets-1 and HTD Identified Small Molecules

### Nanoelectrospray Ionization (NanoESI) Mass Spectrometry of the Recombinant Ets-1 DBD.

Prior to commencing the structure/function investigation of the 15N and/or 15N/13C-labeled Ets-1 DBD domain the recombinant protein is ensured to be monomeric with a molecular mass that is in agreement with the known theoretical mass. These NanoESI mass spectra can be acquired at the Tuft University School of Medicine Core Facility (service fee) on an API QSTAR Pulsar-i quadrapole TOF mass spectrometer in both the positive and negative ion mode. The acquisition and the deconvolution of these data is performed using the AnalystQS Windows PC data system, while offline analysis of these mass spectra is accomplished using Bioanalyst version 1.0 software (Applied Biosystems/MDS Sciex).

### Characterization of Ets-1 Small Molecule Interactions by Isothermal Titration Calorimetry

To further characterize the binding affinity and stoichiometry of the small molecule interactions with the Ets-1 DBD, isothermal titration calorimetry (ITC) is used. ITC is an essential complement to the NMR studies of Ets-1 with HTD identified “hits” since it provides a detailed quantitative description of the binding thermodynamics (155). These ITC measurements are performed on a Microcal VP-ITC titration calorimeter (MicroCal, LLC). Typically, 5-10 μL aliquots of the small molecule in a buffered solution is injected into various concentrations of the Ets-1 DBD in a sample cell of 1.4 mL total volume and the heat of the reaction measured for each aliquot. A series of 20 individual titration points will make up each experiment, and following each titration point, the sample is allowed to equilibrate. All isotherms are corrected for the heat of mixing and/or dilution by subtraction of the isotherm obtained following the injection of the small molecule or Ets-1 DBD into buffer alone. Analysis necessary to deconvolute the data for the best-fit model is performed offline with Origin software (MicroCal, LLC).

### Two-Dimensional 15N-1H Heteronuclear Single Quantum Coherence (HSQC)

NMR spectroscopy is the preferred method for characterizing the structural perturbations resulting from P-DNA and/or P-ligand interactions over several affinity ranges. Ligand-induced localized changes of the chemical environment of nuclei that are within the recognition/binding site result in chemical shift perturbations (CSP) of those resonances involved in binding (91, 96, 156). The ITC experiments provide Kd information for the Ets-1 small molecule complex that will guide our NMR experiments. As is often the case for small molecules the ligand binding exchange rate is likely to be in the fast regime (˜108-109 M) on the NMR chemical shift timescale. In the fast exchange regime these chemical shift data provide an estimate of the dissociation constant, Kd that is easily calculated by correlating binding site chemical shift perturbation as a function of total ligand concentration (157). Should the compounds bind within intermediate exchange rate, data will be collected at increased temperatures and/or field strengths, which helps to resolve the data. Chemical shift perturbation analysis is an integral component of many discovery initiatives, due to the ease of data collection and analysis. These data are invaluable for translating these binding determinants into pharmacophoric descriptors for use in structure-guided HTD exploration, validation and/or optimization initiatives. Using chemical shift perturbation analysis of the amide (15N-1H) resonances we will collect sensitivity-enhanced, 2D 15N-1H heteronuclear single-quantum coherence (HSQC) experiments correlating each nitrogen (15N) nucleus of the Ets-1 DBD to its directly bonded proton (1H). All data will be collected at a proton frequency of 500 MHz on a Varian INOVA 500 MHz spectrometer. Typical sample conditions include; 0.1-0.4 mM 15N-labeled or 15N/13C-labeled Ets-1 (determined using amino acid analysis), 50 mM NaPO4 (pH 6.5), 100 mM NaCl, 10 mM NaS2O4 and 1 mM DSS (internal proton reference) in 90%:10% (H2O:2 H2O). Should it be necessary, we will optimize the sample conditions (salt, buffer, pH, metal-ions and temperature) using micro-drop screening (158) to ensure NMR data of the highest quality is obtained for the Ets-1 DBD protein.

### Deuterium Exchange Experiments

In addition to chemical shift mapping, deuterium exchange can be used to probe intermolecular TF-DNA contacts and gain insight into the conformational flexibility of the complex as was recently used to identify a phosphorylation-dependent conformational perturbation of Ets-1 (121). Slow intrinsic rates of amide proton (NH) exchange for deuterium (NH.ND) are indicative of reduced solvent accessibility and/or imposed structural constraints. The largest protection from proton chemical exchange with deuterium will occur where the protein is buried at the DNA interface or where helices are closely packed against one another. Deuterium exchange experiments will be carried out on lyophilized samples of 15N-labeled ETS-1 DBD alone or from lyophilized samples of Ets-1 in complex with small molecule hits that will be redissolved in D2O (99.99%). Immediately following hydration in D2O, a series of 15N-1H-HSQC spectra will be recorded at regular time intervals. Residue protection factors will be extrapolated from the measured 15N-1H peaks intensities over time and compared with those collected in D2O without the small molecule. These atomic details will provide additional information pertaining to the critical determinants within the Ets-1 DBD that are responsible for interacting with small molecule “hits” and for characterizing the mechanism of action, both of which are important in the optimization stage.

### Ets-1 Small Molecule Co-Complex by NMR Spectroscopy

If our CSP analysis data of Ets-1 monitored at increasing concentrations of our small molecule “hits”, combined with the known structure of Ets-1 do not provide the resolution needed to further characterize the mechanism by which the small molecule modulates Ets-1 activity we will determine the molecule co-complex structure. Briefly, structure determination of biological macromolecules by NMR spectroscopy involves the complete resonance assignments of all (1H, 15N and 13C) atoms made using a suite of two-dimensional (2D) and three-dimensional (3D) triple resonance (1H, 15N and 13C) experiments. Following the identification of each backbone amide proton (1HN) and nitrogen (15N) resonance using the previously detailed 2D HSQC experiment we will use a series of triple resonance experiments (1H, 15N and 13C) to assign each amino acid using sequential 1 bond (1J) and 2 bond (2J) couplings determined from HNCA and HNCO experiments. These sequential backbone assignments are then verified following superimposition of the HNCA spectrum with an HN(CO)CA spectrum, which correlates the 1HN proton of residue (i) with the alpha and carbonyl carbons of the preceding residue, Ca((i-1) and (C′(.−1)), but not its own Ca, ensuring that the interresidue and intraresidue Ca carbons are correctly assigned. This triple resonance sequential assignment approach identifies the connectivities between neighboring spin systems but does not identify specific amino acid types. Specific amino acids are then assigned using a series of triple resonance experiments that correlate the backbone atoms with side chain atoms including: the CBCA(CO)NH and HCCH-TOCSY experiments. AUTOASSIGN and Aria two automated assignment programs that rely upon uniform isotopic labeling strategies (13C and 15N) to provide an efficient approach for much of the assignment strategy (159, 160). Intermolecular and intraprotein distance restraints will be obtained from a series of 3D 15N- and 13C nuclear Overhauser effect spectroscopy (NOESY) data. Taken together these data will be used to generate a restraint file for use in calculating a family of structures using a combination of distance geometry and simulated annealing, DGII (InsightII, Accelrys) with a CVFF forcefield or CNS (161). Of note for structural calculations we will fix the protein structure with the exception of those residues that form the small molecule binding site as measured from our HS QC CSP experiments (10).

### V: COMPOUNDS IDENTIFIED BY THE METHODS OF THE PRESENT INVENTION AND THEIR USE

Compounds (candidate agents) that have been identified by the methods of the present invention and can be used for treatment of inflammation and angiogenisis related diseases and/or disorders are represented (in certain embodiments) by the formula A-D, in which A is a moiety capable of interacting with a DNA binding domain of a transcription factor, and D is a moiety capable of interacting with a nucleic acid to which the transcription factor binds;

wherein the compound is capable of modulating the ability of the transcription factor to bind to the nucleic acid, and pharmaceutically acceptable esters, salts, and prodrugs thereof. It has been reported that several amino acids are preferentially localized at the TF-DNA interface of the ETS family including; Arg, Lys, Asn, Gln and aromatic residues. This cluster of residues functions as a tactile sensor that undergoes DNA-dependent conformational changes that are used to distinguish between DNA sequences, providing an additional level of specificity. Accordingly, in certain embodiments, the compounds of the invention are capable of binding to a TF-DNA interface of a transcription factor of the ETS family.

The compounds can be non-peptidic. In certain embodiments, the A moiety or the D moiety can be an aromatic group, an optionally substituted phenyl group, an optionally substituted heteroaryl group wherein the heteroaryl group is an optionally substituted purine group. In certain embodiments, the A moiety or the D moiety is a hydrogen bond donor.

Other compounds that can be used in the methods of treatment of the present invention can selectively bind to a loop region of Ets-1 (e.g., a loop region of Ets-1 hich contains residues or sequences characteristic of Ets-1) or do not bind in the major groove of the nucleic acid or bind in a binding pocket defined by the nucleic acid and a loop region of Ets-1.

The compounds for use in the treatment methods of the present invention can be selected from the group consisting of:

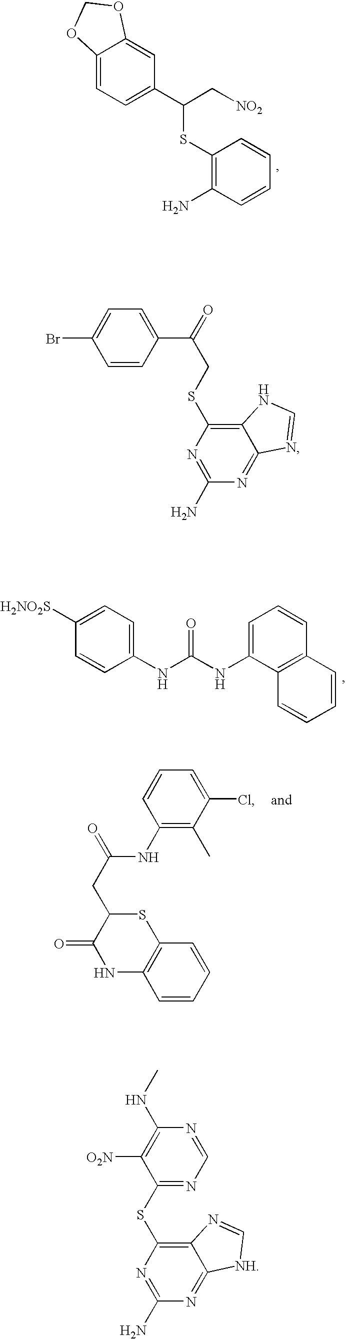

Also included are the pharmaceutically-acceptable esters, salts, and prodrugs of these compounds.

### VI: PHARMACEUTICAL COMPOSITIONS

The compounds identified by the methods of the present invention and described above (also referred to herein as “candidate agents”) of the invention can be incorporated into pharmaceutical compositions suitable for administration. Such compositions typically comprise the active compounds and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. As discussed above, supplementary active compounds can also be incorporated into the compositions.

A pharmaceutical composition of the invention is formulated to be compatible with its intended route of administration. Examples of routes of administration include, but are not limited to, parenteral, e.g., intravenous, intradermal, intramuscular, intraosseous, subcutaneous, oral, intranasal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition preferably is sterile and should be fluid to the extent that easy syringability exists. The compositions suitably should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in a therapeutically effective or beneficial amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. Suitable oral compositions may be e.g. enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as hydroxyfluoroalkane (HFA), or a nebulizer. Alternatively, intranasal preparations may be comprised of dry powders with suitable propellants such as HFA.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially e.g. from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED50 (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from the cell culture assays and animal studies can be used in formulating a range of dosage for use in humans. The dosage of such compounds lies preferably within a range of circulating concentrations that include the ED50 with little or no toxicity. The dosage may vary within—this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions (e.g. written) for administration, particularly such instructions for use of the active agent to treat against a disorder or disease as disclosed herein, including diseases or disorders associated with transcription factors, particularly Est-1.

All documents mentioned herein are expressly incorporated herein by reference in their entirety.

### VII: EXEMPLIFICATION

The present invention is further illustrated by the following Examples. The Examples are provided to aid in the understanding of the invention and are not construed as a limitation thereof. All examples are carried out using standard techniques, which are well known and routine to those of skill in the art, except where otherwise described in detail. Routine molecular biology techniques of the following examples can be carried out as described in standard laboratory manuals.

### Example 1

### Peptide Inhibitors of ETS TF ELF-1 and NRF

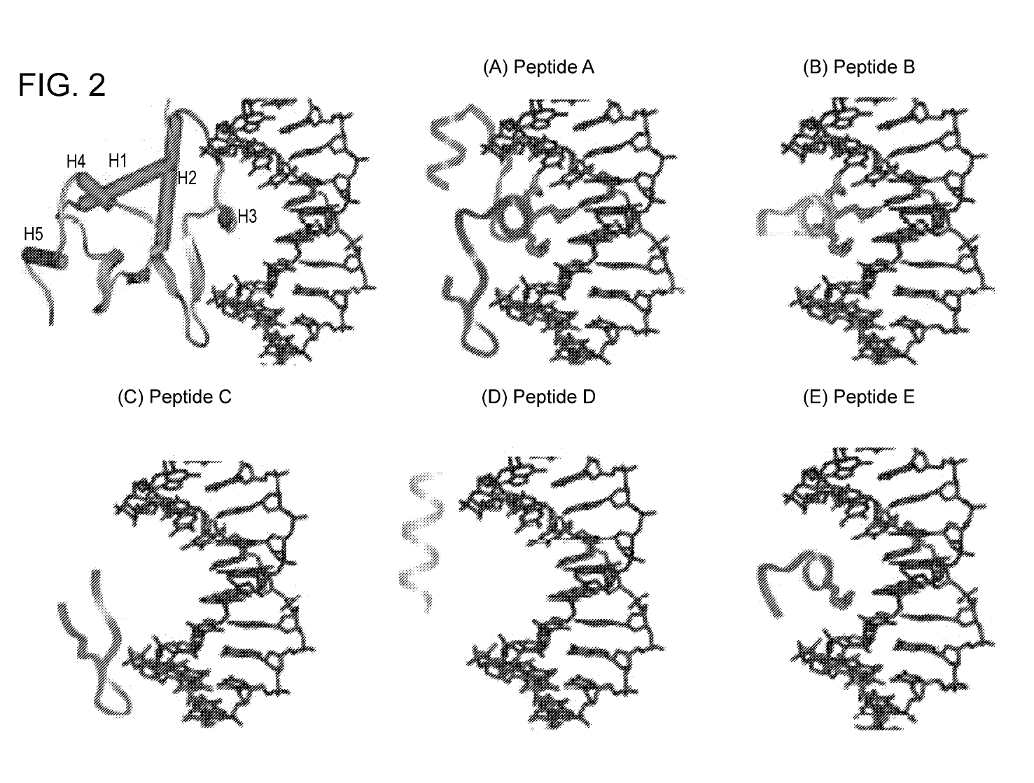

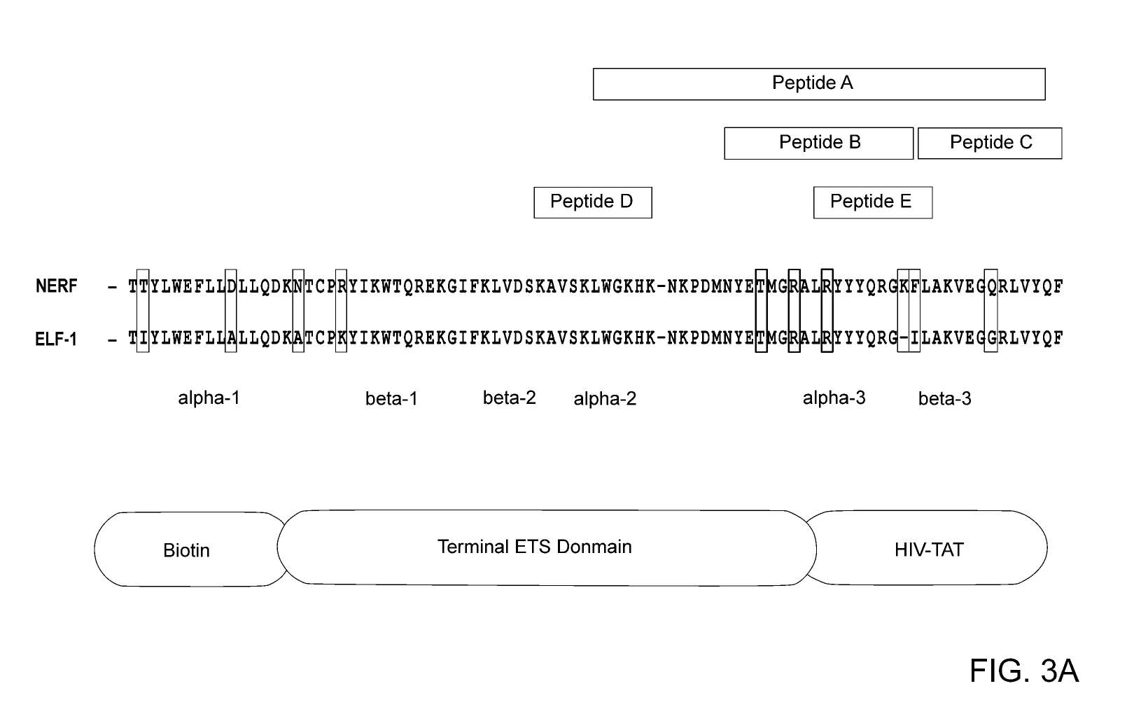

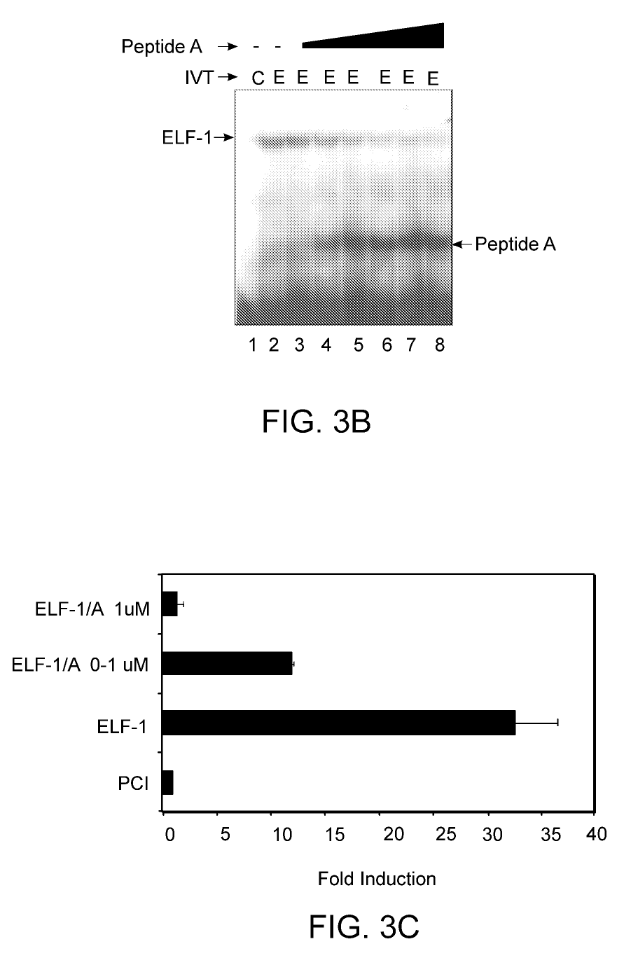

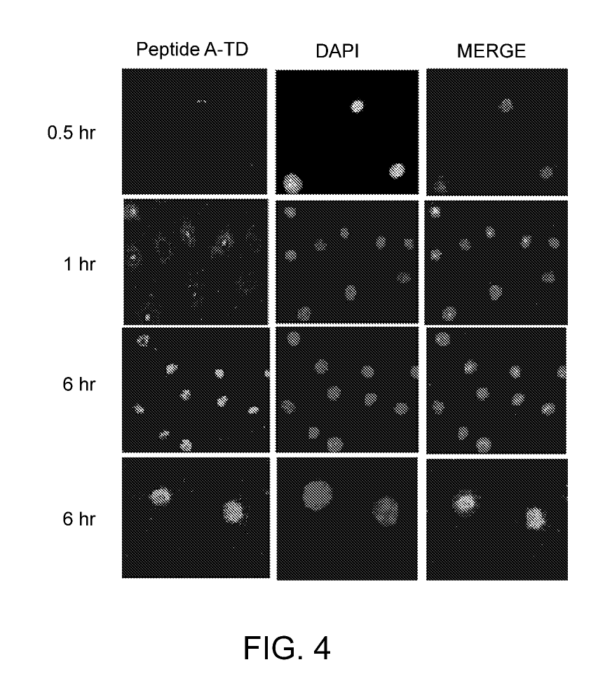

Several recent studies have demonstrated the importance of peptide inhibitors as novel tools in cell biology for understanding and further characterizing the signaling mechanisms mediated by transient P—P and TF-DNA interactions (102). These peptidic approaches have demonstrated significant success, showing therapeutic promise in several disease indications including inflammation and autoimmune disease (1, 103-105). The DBD of the ETS family of proteins is highly conserved at the primary sequence and secondary structure level. However, the three-dimensional structures of ETS family members are comprised of both structurally conserved regions as well as some unique notable structural differences that are believed responsible for substrate FIG. 2. Using the Xray structure of Ets-1 DBD in complex with its high affinity core sequence, we synthesized ETSpeptides (A-E) and assessed their ability to block ETS/DNA gel mobility shift and transactivation assays (1). recognition of the flanking regions within the EBS [(2, 29, 39) and references therein]. To understand if isolated fragments of the ETS domain that include DNA contact residues located in the recognition helix (H3 or alpha-3) could abrogate DNA binding and transactivation, we chemically synthesized several peptides [FIG. 2 (A-E)]. These peptides are 10-34 amino acids in length and correspond to sequentially juxtaposed regions within the highly conserved ETS domain of ETS factors NERF2 and ELF-1 (FIG. 2) (53). Using the crystal structure of the Ets-1 DBD in complex with DNA that was solved by Garvie and coworkers to a resolution of 2.4 Å as a model, we have illustrated the theoretical secondary structure and sequential position of these peptides [FIG. 3A and FIG. 2 (A-E)]. We have evaluated the ability of these synthetic ETS peptides to compete with and/or block DNA binding in EMSAs and reporter gene transactivation assays (FIGS. 3B and 3C). Only peptide A, which is 34 amino acids in length and includes the recognition helix (H3 or alpha-3) (22), a portion of H2 and the first two strands of the β sheet was able to block ELF-1 binding to the EBS's in the Tie2 promoter. This peptide was also able to potently block transactivation of the Tie2 promoter in a dose dependent manner (1). To verify the specificity of this interaction we synthesized a peptide variant in which three amino acids at positions Thr388, Arg391 and Arg394 (T388R391R394, rendered and illustrated in blue (FIG. 2)) that are required for mediating the DNA major groove interaction of the ETS domain were mutated. Mutation of these amino acids to glycine resulted in a peptide that did not block ELF-1 binding to DNA (1). The addition of the HIV-TAT protein-transducing domain to a peptide facilitated peptide transduction across the membrane of human primary endothelial cells and interestingly nuclear accumulation of the peptide in endothelial cells (FIG. 4). This resulted in demonstrable angiogenesis repression through the inhibition of tube formation from endothelial cells plated on matrigel (1). In addition, Peptide A but not the mutant (T388R391R394/GGG) peptide inhibited B16 melanoma tumor growth through marked reductions in blood vessel density (1). Although these peptides provide novel tools for targeting transcription factors they also provide critical pharmacophoric details pertaining to those determinants within the TF-DNA interface that mediate this interaction. Within this proposal we will use these critical pharmacophoric descriptors to selective identify small molecules that target this TF-DNA interface and are capable of dissecting the regulatory networks mediated by the Ets-1 in complex with it cognate DNA sequence.

### Example 2

### HTD Identification of Small Molecules Targeting the Ets-1 DNA Interface

Our preliminary data demonstrating that small molecule inhibitors of the TF-DNA interface of Ets-1 could be identified from publicly accessible chemical repositories virtually screened using HTD provides preliminary proof concept data. Ets-1 is an excellent target for this approach due to its critical role in inducing the expression of a number of genes involved in VSMC growth and proliferation, endothelial cell activation vascular inflammation and cancer ((16, 17, 106) and references therein). Furthermore, we chose this target due to the extensive structural knowledge provided by several crystal structures of Ets-1 in complex with different DNA sequences and/or protein partners (2). In defining the Ets-1 “hotspot” or cavity to be targeted in our HTD studies we actually identified two plausible TF-DNA interfaces that were amenable to this approach (FIG. 5A, white arrows). Due to the sequence divergence within the right TF-DNA interfacial cavity and the presence of critical TF-DNA contacts (FIG. 5, right arrow) our preliminary, proof of concept data have been generated using this interface. The entire TF-DNA interface is illustrated as a red separated surface (FIG. 5A) and each of the targeted cavities is similar in size (−500-700 Å) to many P—P interfaces currently being exploited using HTD and they are comprised of hydrophobic properties, which is ideal for docking small molecules with preferential lipophilicity as well as optimizing the selection of compounds that obey the Lipinsky “rule of five” (107-109). The TF-DNA interface being targeted (illustrated as a translucent Connolly surface, FIG. 5B) is comprised of residues from the canonical third helix, H3, which anchors Ets-1 to the DNA major groove as well as residues that are unique Ets-1 but are involved minor groove interactions at the 3′ end of the EBS, thus contributing to DNA binding specificity. Using structure-based HTD we have identified 27 “hits” from the NCI Diversity Set of 1990 molecules, which we filtered for “rule of 5” compliant molecules. The hit list was scored using several scoring functions, however ChemScore (110) provided reproducible hit scoring and ranking. In determining the most reproducible scoring function we are using Neural Network Analysis (NNA) for learning set evaluation that permits 80-90% of the database to be eliminated with a confidence level of 95% in collaboration with Dr Xavier Morelli (http://gfscore.cnrs-mrs.fr) who has extensive experience in applying NAA to P—P interactions. One of our NCI “hits”, compound #28, which is docked in its preferential pose in FIG. 5C inhibits the Ets-1-DNA interaction in the low uM range as estimated using ESMAs. We have recently demonstrated that this compound inhibits transactivation assays 8 fold and decreases Ets-1 mediated gene expression 50-60% in HEK293 cells (data not shown). We have performed similar HTD calculations for several other ETS family members, employing critical descriptors that are unique to each ETS structure and have identified ˜30 unique small molecules for each target (data not shown).

### Example 3

### Cloning, Expression and Purification of Ets-1 DBD for Structural Validation of HTD “Hits”

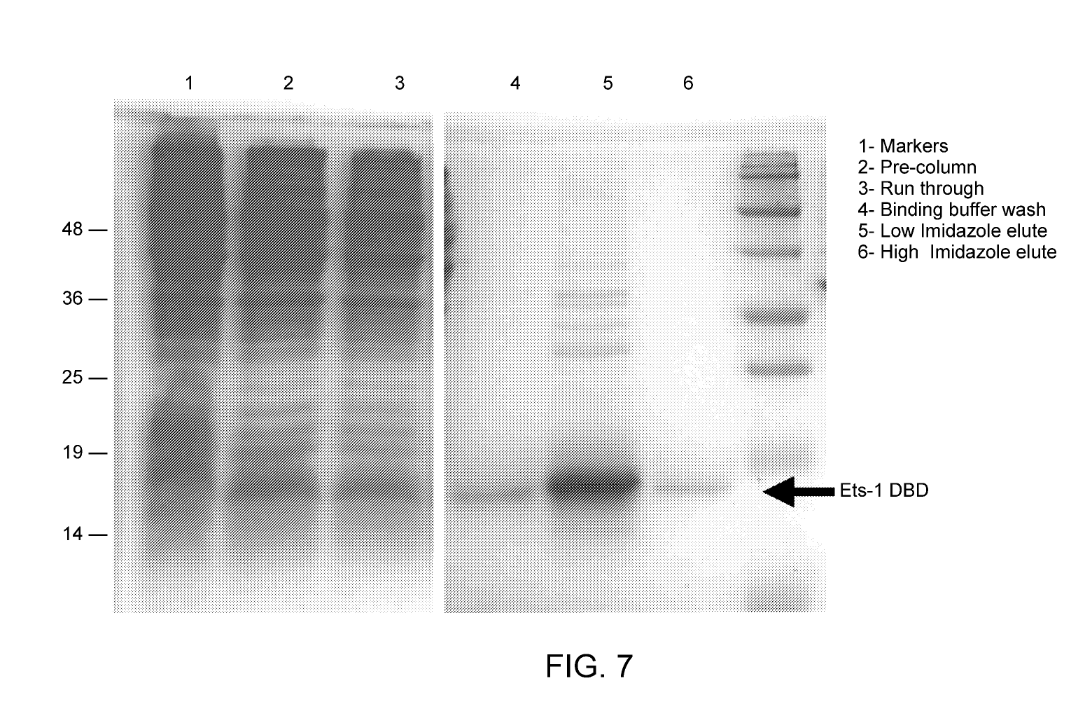

NMR provides a robust platform to characterize both the ligand binding site and affinity, while simultaneously providing a window through which the entire target protein or proteins can be structurally observed without the need of an assay to detect this interaction (96, 99). Many of the important advances in the use of NMR in small molecule discovery and optimization have been and continue to be reliant on the recombinant expression of target proteins in E. coli bacterial expression systems to facilitate the preparation of adequate quantities of isotopically enriched target protein. To characterize the mechanism of action of the small molecule modulators identified using HTD, we have recombinantly expressed the DBD of Ets-1. Briefly, a gene insert for the human Ets-1 DNA binding domain (DBD), residues Ile335-Ser420, has been PCR amplified from full-length human Ets-1 cDNA using primers designed to facilitate directional cloning into the pET-32b expression vector (Novagen). The Ets-1 DBD sequence was inserted downstream of a thioredoxin fusion sequence comprised, a six-residue histidine affinity tag and an enterokinase (EK) recognition site in pET-32b. Ligated vector containing the Ets-1 DBD gene insert was used to 1 2 3 4 5 6 2-Pre-column transform chemically competent E. coli BL21 (DE3) cells (Novagen). Single colony transformants were screened for the Ets-1 DBD coding 48 insert by PCR using primers complementary to adjacent plasmid25 sequences. Overnight cultures of positive isolates were grown at 37° C. in LB media containing carbenicilin to prepare glycerol stocks and to isolate 14 plasmid DNA using Qiaquick Miniprep columns (Qiagen) for sequence verification. The expression and subsequent nickel column purification of the Ets-1 DBD fusion protein is shown in FIG. 7, by SDS PAGE. We have optimized the experimental conditions for EK cleavage (data not shown), to generate the Ets-1 DBD protein alone.

### Example 4

### In Vitro Evaluation of Ets-1 Efficacy and Specificity

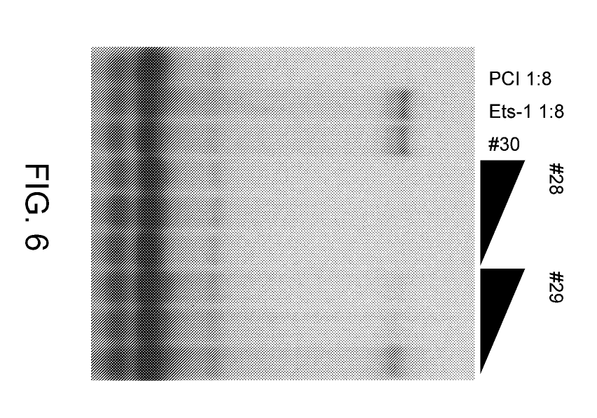

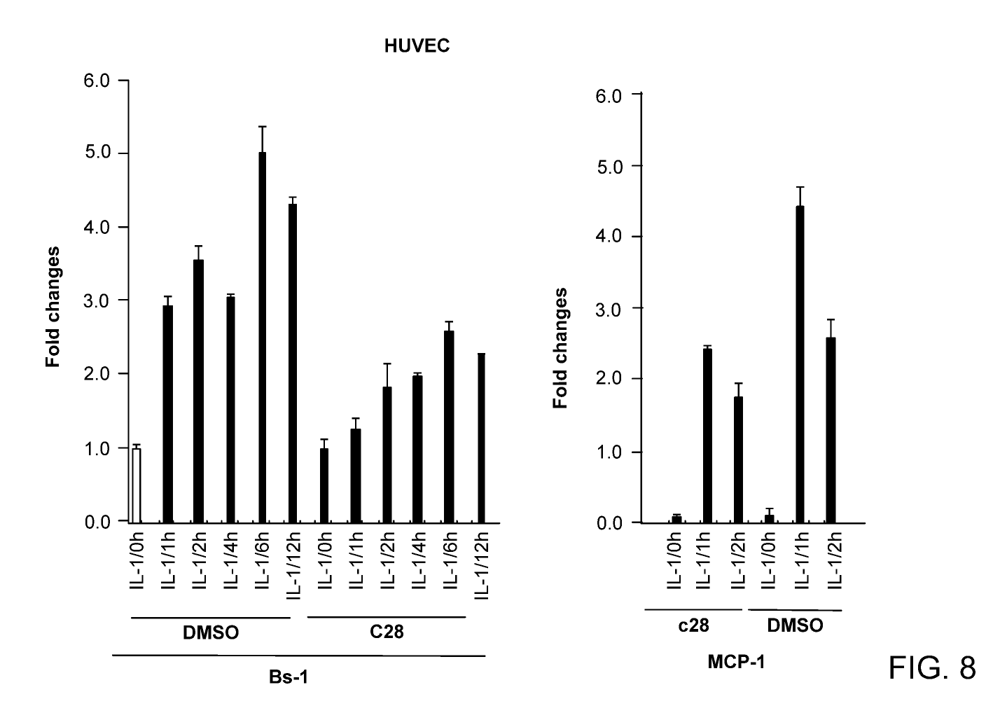

Human umbilical vein endothelial cells (HUVECs) were preincubated with 10 μM of our NCI “hit” compound (#28, NCI-371777, 2-{[1-(1,3-benzodioxol-5-yl)-2-nitroethyl]thio}aniline), that we have demonstrated inhibits Ets-1 DNA binding (FIG. 6), and an equivalent amount of DMSO (vehicle) for 1 h prior to addition of the inflammatory cytokine Interleukin 1 (IL-1) (10 ng/ml). Total RNA was isolated for real time PCR to measure Ets-1 expression levels as well as the IL-1 inducible, Ets-1 dependent MCP-1 expression levels, following stimulation for 1, 2, 4, 6 and 12 hours. IL-1 induced Ets-1 expression ˜4 fold at 2 hr, however compound #28 reduced Ets-1 induction by greater than 50% during this time frame. This is consistent with the known positive auto-regulation of Ets-1 gene expression by Ets-1 itself. Similarly, induction of MCP-1 was induced up to 45 fold by the addition of the cytokine IL-1, and again FIG. 8. compound #28 reduced MCP-1 expression by greater than 40%. These results clearly support that compound 28 is able to selectively regulate IL-1 inducible genes that are dependent on Ets-1 transactivation in HUVEC cells.

### REFERENCES

The following specific references, also incorporated by reference, are generally indicated in the examples and discussion above by a number in parentheses.
1. Huang, X., Brown, C., Ni, W., Maynard, E., Rigby, A. C., and Oettgen, P. Critical role for the Ets transcription factor ELF-1 in the development of tumor angiogenesis. Blood, 2005.
2. Garvie, C. W., Hagman, J., and Wolberger, C. Structural studies of Ets-1/Pax5 complex formation on DNA. Mol Cell, 8: 1267-1276, 2001.
3. Kong, D., Park, E. J., Stephen, A. G., Calvani, M., Cardellina, J. H., Monks, A., Fisher, R. J., Shoemaker, R. H., and Melillo, G. Echinomycin, a small-molecule inhibitor of hypoxia-inducible factor-1 DNA-binding activity. Cancer Res, 65: 9047-9055, 2005.
4. Melnick, A. M., Adelson, K., and Licht, J. D. The theoretical basis of transcriptional therapy of cancer: can it be put into practice? J Clin Oncol, 23: 3957-3970, 2005.
5. Arkin, M. R., Randal, M., DeLano, W. L., Hyde, J., Luong, T. N., Oslob, J. D., Raphael, D. R., Taylor, L., Wang, J., McDowell, R. S., Wells, J. A., and Braisted, A. C. Binding of small molecules to an adaptive protein-protein interface. Proc Natl Acad Sci USA, 100: 1603-1608, 2003.
6. Arkin, M. R. and Wells, J. A. Small-molecule inhibitors of protein-protein interactions: progressing towards the dream. Nat Rev Drug Discov, 3: 301-317, 2004.
7. Lee, Y. S., Bergson, P., He, W. S., Mrksich, M., and Tang, W. J. Discovery of a small molecule that inhibits the interaction of anthrax edema factor with its cellular activator, calmodulin. Chem Biol, 11: 1139-1146, 2004.
8. Berg, T., Cohen, S. B., Desharnais, J., Sonderegger, C., Maslyar, D. J., Goldberg, J., Boger, D. L., and Vogt, P. K. Small-molecule antagonists of Myc/Max dimerization inhibit Myc-induced transformation of chicken embryo fibroblasts. Proc Natl Acad Sci U S A, 99: 3830-3835, 2002.
9. Best, J. L., Amezcua, C. A., Mayr, B., Flechner, L., Murawsky, C. M., Emerson, B., Zor, T., Gardner, K. H., and Montminy, M. Identification of small-molecule antagonists that inhibit an activator: coactivator interaction. Proc Natl Acad Sci USA, 101: 17622-17627, 2004.
10. Oltersdorf, T., Elmore, S. W., Shoemaker, A. R., Armstrong, R. C., Augeri, D. J., Belli, B. A., Bruncko, M., Deckwerth, T. L., Dinges, J., Hajduk, P. J., Joseph, M. K., Kitada, S., Korsmeyer, S. J., Kunzer, A. R., Letai, A., Li, C., Mitten, M. J., Nettesheim, D. G., Ng, S., Nimmer, P. M., O'Connor, J. M., Oleksijew, A., Petros, A. M., Reed, J. C., Shen, W., Tahir, S. K., Thompson, C. B., Tomaselli, K. J., Wang, B., Wendt, M. D., Zhang, H., Fesik, S. W., and Rosenberg, S. H. An inhibitor of Bcl-2 family proteins induces regression of solid tumours. Nature, 435: 677-681, 2005.
11. Vassilev, L. T., Vu, B. T., Graves, B., Carvajal, D., Podlaski, F., Filipovic, Z., Kong, N., Kammlott, U., Lukacs, C., Klein, C., Fotouhi, N., and Liu, E. A. In vivo activation of the p53 pathway by small-molecule antagonists of MDM2. Science, 303: 844-848, 2004.
12. Olenyuk, B. Z., Zhang, G. J., Klco, J. M., Nickols, N. G., Kaelin, W. G., Jr., and Dervan, P. B. Inhibition of vascular endothelial growth factor with a sequence-specific hypoxia response element antagonist. Proc Natl Acad Sci USA, 101: 16768-16773, 2004.
13. Rishi, V., Potter, T., Laudeman, J., Reinhart, R., Silvers, T., Selby, M., Stevenson, T., Krosky, P., Stephen, A. G., Acharya, A., Moll, J., Oh, W. J., Scudiero, D., Shoemaker, R. H., and Vinson, C. A high-throughput fluorescence-anisotropy screen that identifies small molecule inhibitors of the DNA binding of B-ZIP transcription factors. Anal Biochem, 340: 259-271, 2005.
14. Sharrocks, A. D. The ETS-domain transcription factor family. Nat Rev Mol Cell Biol, 2: 827-837, 2001.
15. Sharrocks, A. D. Complexities in ETS-domain transcription factor function and regulation: lessons from the TCF (ternary complex factor) subfamily. The Colworth Medal Lecture. Biochem Soc Trans, 30:1-9, 2002.
16. Seth, A. and Watson, D. K. ETS transcription factors and their emerging roles in human cancer. Eur J Cancer, 41: 2462-2478, 2005.
17. Oikawa, T. ETS transcription factors: possible targets for cancer therapy. Cancer Sci, 95: 626-633, 2004.
18. Kodandapani, R., Pio, F., Ni, C. Z., Piccialli, G., Klemsz, M., McKercher, S., Maki, R. A., and Ely, K. R. A new pattern for helix-turn-helix recognition revealed by the PU.1 ETS-domain-DNA complex. Nature, 380: 456-460, 1996.
19. Dittmer, J. and Nordheim, A. Ets transcription factors and human disease. Biochim Biophys Acta, 1377: F1-11, 1998.
20. Oikawa, T. and Yamada, T. Molecular biology of the Ets family of transcription factors. Gene, 303: 1134, 2003.
21. Dittmer, J. The biology of the Ets1 proto-oncogene. Mol Cancer, 2: 29, 2003.
22. Luscombe, N. M., Austin, S. E., Berman, H. M., and Thornton, J. M. An overview of the structures of protein-DNA complexes. Genome Biol, 1: REVIEWS001, 2000. 16.
23. Luscombe, N. M., Laskowski, R. A., and Thornton, J. M. Amino acid-base interactions: a three-dimensional analysis of protein-DNA interactions at an atomic level. Nucleic Acids Res, 29: 2860-2874, 2001.
24. Luscombe, N. M. and Thornton, J. M. Protein-DNA interactions: amino acid conservation and the effects of mutations on binding specificity. J Mol Biol, 320: 991-1009, 2002.
25. Werner, M. H., Clore, G. M., Fisher, C. L., Fisher, R. J., Trinh, L., Shiloach, J., and Gronenborn, A. M. Correction of the NMR structure of the ETS1/DNA complex. J Biomol NMR, 10: 317-328, 1997.
26. Werner, M. H., Bianchi, M. E., Gronenborn, A. M., and Clore, G. M. NMR spectroscopic analysis of the DNA conformation induced by the human testis determining factor SRY. Biochemistry, 34: 1199812004, 1995.
27. Liang, H., Mao, X., Olejniczak, E. T., Nettesheim, D. G., Yu, L., Meadows, R. P., Thompson, C. B., and Fesik, S. W. Solution structure of the ets domain of Fli-1 when bound to DNA. Nat Struct Biol, 1: 871875, 1994.
28. Batchelor, A. H., Piper, D. E., de la Brousse, F. C., McKnight, S. L., and Wolberger, C. The structure of GABPalpha/beta: an ETS domain—ankyrin repeat heterodimer bound to DNA. Science, 279: 10371041, 1998.
29. Wang, Y., Feng, L., Said, M., Balderman, S., Fayazi, Z., Liu, Y., Ghosh, D., and Gulick, A. M. Analysis of the 2.0 A crystal structure of the protein-DNA complex of the human PDEF Ets domain bound to the prostate specific antigen regulatory site. Biochemistry, 44: 7095-7106, 2005. 30.
Obika, S., Reddy, S. Y., and Bruice, T. C. Sequence specific DNA binding of Ets-1 transcription factor: molecular dynamics study on the Ets domain—DNA complexes. J Mol Biol, 331: 345-359, 2003.
31. Tootle, T. L. and Rebay, I. Post-translational modifications influence transcription factor activity: a view from the ETS superfamily. Bioessays, 27: 285-298, 2005.
32. Graves, B. J. and Petersen, J. M. Specificity within the ets family of transcription factors. Adv Cancer Res, 75: 1-55, 1998.
33. Donaldson, L. W., Petersen, J. M., Graves, B. J., and McIntosh, L. P. Solution structure of the ETS domain from murine Ets-1: a winged helix-turn-helix DNA binding motif. Embo J, 15: 125-134, 1996.
34. Petersen, J. M., Skalicky, J. J., Donaldson, L. W., McIntosh, L. P., Alber, T., and Graves, B. J. Modulation of transcription factor Ets-1 DNA binding: DNA-induced unfolding of an alpha helix. Science, 269: 1866-1869, 1995.
35. Bassuk, A. G. and Leiden, J. M. The role of Ets transcription factors in the development and function of the mammalian immune system. Adv Immunol, 64: 65-104, 1997.
36. Brennan, R. G. The winged-helix DNA-binding motif: another helix-turn-helix takeoff. Cell, 74: 773-776, 1993.
37. Mo, Y., Vaessen, B., Johnston, K., and Marmorstein, R. Structures of SAP-1 bound to DNA targets from the E74 and c-fos promoters: insights into DNA sequence discrimination by Ets proteins. Mol Cell, 2: 201-212, 1998.
38. Mo, Y., Vaessen, B., Johnston, K., and Marmorstein, R. Structure of the elk-1-DNA complex reveals how DNA-distal residues affect ETS domain recognition of DNA. Nat Struct Biol, 7: 292-297, 2000.
39. Garvie, C. W., Pufall, M. A., Graves, B. J., and Wolberger, C. Structural analysis of the autoinhibition of Ets-1 and its role in protein partnerships. J Biol Chem, 277: 45529-45536, 2002.
40. Pufall, M. A. and Graves, B. J. Ets-1 flips for new partner Pax-5. Structure (Camb), 10: 11-14, 2002.
41. Buchwalter, G., Gross, C., and Wasylyk, B. Ets ternary complex transcription factors. Gene, 324: 1-14, 2004.
42. Risau, W. Differentiation of endothelium. Faseb J, 9: 926-933, 1995.
43. Risau, W. and Flamme, I. Vasculogenesis. Annu Rev Cell Dev Biol, 11: 73-91, 1995.
44. Hasegawa, Y., Abe, M., Yamazaki, T., Niizeki, O., Shiiba, K., Sasaki, I., and Sato, Y. Transcriptional regulation of human angiopoietin-2 by transcription factor Ets-1. Biochem Biophys Res Commun, 316: 52-58, 2004.
45. Valter, M. M., Hugel, A., Huang, H. J., Cavenee, W. K., Wiestler, O. D., Pietsch, T., and Wernert, N. Expression of the Ets-1 transcription factor in human astrocytomas is associated with Fms-like tyrosine kinase-1 (Flt-1)/vascular endothelial growth factor receptor-1 synthesis and neoangiogenesis. Cancer Res, 59: 5608-5614, 1999.
46. Valter, M. M., Wiestler, O. D., and Pietsche, T. Differential control of VEGF synthesis and secretion in human glioma cells by IL-1 and EGF. Int J Dev Neurosci, 17: 565-577, 1999.
47. Ikeda, T., Wakiya, K., and Shibuya, M. Characterization of the promoter region for flt-1 tyrosine kinase gene, a receptor for vascular endothelial growth factor. Growth Factors, 13: 151-162, 1996. 17
48. Wakiya, K., Begue, A., Stehelin, D., and Shibuya, M. A cAMP response element and an Ets motif are involved in the transcriptional regulation of flt-1 tyrosine kinase (vascular endothelial growth factor receptor 1) gene. J Biol Chem, 271: 30823-30828, 1996.
49. Zhan, Y., Brown, C., Maynard, E., Anshelevich, A., Ni, W., Ho, I. C., and Oettgen, P. Ets-1 is a critical regulator of Ang II-mediated vascular inflammation and remodeling. J Clin Invest, 115: 2508-2516, 2005.
50. Deshayes, K., Schaffer, M. L., Skelton, N. J., Nakamura, G. R., Kadkhodayan, S., and Sidhu, S. S. Rapid identification of small binding motifs with high-throughput phage display: discovery of peptidic antagonists of IGF-1 function. Chem Biol, 9: 495-505, 2002.
51. Sidhu, S. S., Lowman, H. B., Cunningham, B. C., and Wells, J. A. Phage display for selection of novel binding peptides. Methods Enzymol, 328: 333-363, 2000.
52. Sidhu, S. S., Bader, G. D., and Boone, C. Functional genomics of intracellular peptide recognition domains with combinatorial biology methods. Curr Opin Chem Biol, 7: 97-102, 2003.
53. Turkson, J., Kim, J. S., Zhang, S., Yuan, J., Huang, M., Glenn, M., Haura, E., Sebti, S., Hamilton, A. D., and Jove, R. Novel peptidomimetic inhibitors of signal transducer and activator of transcription 3 dimerization and biological activity. Mol Cancer Ther, 3: 261-269, 2004.
54. Turkson, J., Ryan, D., Kim, J. S., Zhang, Y., Chen, Z., Haura, E., Laudano, A., Sebti, S., Hamilton, A. D., and Jove, R. Phosphotyrosyl peptides block Stat3-mediated DNA binding activity, gene regulation, and cell transformation. J Biol Chem, 276: 45443-45455, 2001.
55. Tournaire, R., Simon, M. P., le Noble, F., Eichmann, A., England, P., and Pouyssegur, J. A short synthetic peptide inhibits signal transduction, migration and angiogenesis mediated by Tie2 receptor. EMBO Rep, 5: 262-267, 2004.
56. Zhang, L., Torgerson, T. R., Liu, X. Y., Timmons, S., Colosia, A. D., Hawiger, J., and Tam, J. P. Preparation of functionally active cell-permeable peptides by single-step ligation of two peptide modules. Proc Natl Acad Sci USA, 95: 9184-9189, 1998.
57. Futaki, S., Suzuki, T., Ohashi, W., Yagami, T., Tanaka, S., Ueda, K., and Sugiura, Y. Arginine-rich peptides. An abundant source of membrane-permeable peptides having potential as carriers for intracellular protein delivery. J Biol Chem, 276: 5836-5840, 2001.
58. Lerman, L. S. Structural considerations in the interaction of DNA and acridines. J Mol Biol, 3: 18-30, 1961.
59. Lerman, L. S. The structure of the DNA-acridine complex. Proc Natl Acad Sci USA, 49: 94-102, 1963.
60. Waring, M. J. and Wakelin, L. P. Echinomycin: a bifunctional intercalating antibiotic. Nature, 252: 653657, 1974.
61. Kopka, M. L., Yoon, C., Goodsell, D., Pjura, P., and Dickerson, R. E. The molecular origin of DNA-drug specificity in netropsin and distamycin. Proc Natl Acad Sci USA, 82: 1376-1380, 1985.
62. Nadassy, K., Wodak, S. J., and Janin, J. Structural features of protein-nucleic acid recognition sites. Biochemistry, 38: 1999-2017, 1999.
63. Sathyapriya, R. and Vishveshwara, S. Interaction of DNA with clusters of amino acids in proteins. Nucleic Acids Res, 32: 4109-4118, 2004.
64. Shimogawa, H., Kwon, Y., Mao, Q., Kawazoe, Y., Choi, Y., Asada, S., Kigoshi, H., and Uesugi, M. A wrench-shaped synthetic molecule that modulates a transcription factor-coactivator interaction. J Am Chem Soc, 126: 3461-3471, 2004.
65. Carbone, G. M., Napoli, S., Valentini, A., Cavalli, F., Watson, D. K., and Catapano, C. V. Triplex DNA-mediated downregulation of Ets2 expression results in growth inhibition and apoptosis in human prostate cancer cells. Nucleic Acids Res, 32: 4358-4367, 2004.
66. Dervan, P. B. and Burli, R. W. Sequence-specific DNA recognition by polyamides. Curr Opin Chem Biol, 3: 688-693, 1999.
67. White, S., Szewczyk, J. W., Turner, J. M., Baird, E. E., and Dervan, P. B. Recognition of the four Watson-Crick base pairs in the DNA minor groove by synthetic ligands. Nature, 391: 468-471, 1998.
68. Wade, W. S., Mrksich, M., and Dervan, P. B. Binding affinities of synthetic peptides, pyridine-2carboxamidonetropsin and 1-methylimidazole-2-carboxamidonetropsin, that form 2:1 complexes in the minor groove of double-helical DNA. Biochemistry, 32: 11385-11389, 1993.
69. Oakley, M. G., Mrksich, M., and Dervan, P. B. Evidence that a minor groove-binding peptide and a major groove-binding protein can simultaneously occupy a common site on DNA. Biochemistry, 31: 10969-10975, 1992.
70. Ghosh, I. and Chmielewski, J. A beta-sheet peptide inhibitor of E47 dimerization and DNA binding. Chem Biol, 5: 439-445, 1998. 18
71. Dervan, P. B. Molecular recognition of DNA by small molecules. Bioorg Med Chem, 9: 2215-2235, 2001.
72. Huth, J. R., Yu, L., Collins, I., Mack, J., Mendoza, R., Isaac, B., Braddock, D. T., Muchmore, S. W., Comess, K. M., Fesik, S. W., Clore, G. M., Levens, D., and Hajduk, P. J. NMR-driven discovery of benzoylanthranilic acid inhibitors of far upstream element binding protein binding to the human oncogene c-myc promoter. J Med Chem, 47: 4851-4857, 2004.
73. Kitchen, D. B., Decornez, H., Furr, J. R., and Bajorath, J. Docking and scoring in virtual screening for drug discovery: methods and applications. Nat Rev Drug Discov, 3: 935-949, 2004.
74. LIPINSKI, C. A. H., A. Navigating chemical space for biology and medicine. Nature, 432: 855-861, 2004.
75. Wu, X., Glickman, J. F., Bowen, B. R., and Sills, M. A. Comparison of assay technologies for a nuclear receptor assay screen reveals differences in the sets of identified functional antagonists. J Biomol Screen, 8: 381-392, 2003.
76. Stockwell, B. R. Exploring biology with small organic molecules. Nature, 432: 846-854, 2004.
77. Swinney, D. C. Biochemical mechanisms of drug action: what does it take for success? Nat Rev Drug Discov, 3: 801-808, 2004.
78. Drews, I. I. Drug discovery today—and tomorrow. Drug Discov Today, 5: 2-4, 2000.
79. Drews, J. Drug discovery: a historical perspective. Science, 287: 1960-1964, 2000.
80. Drews, J. and Ryser, S. The role of innovation in drug development. Nat Biotechnol, 15: 1318-1319, 1997.
81. Hopkins, A. L. and Groom, C. R. The druggable genome. Nat Rev Drug Discov, 1: 727-730, 2002.
82. Fauman, E. B., Hopkins, A. L., and Groom, C. R. Structural bioinformatics in drug discovery. Methods Biochem Anal, 44: 477-497, 2003.
83. Hopkins, A. L. and Groom, C. R. Target analysis: a priori assessment of druggability. Ernst Schering Res Found Workshop 11-17, 2003.
84. Hol, W. G. Structural genomics for science and society. Nat Struct Biol, 7 Suppl: 964-966, 2000.
85. Abola, E., Kuhn, P., Earnest, T., and Stevens, R. C. Automation of X-ray crystallography. Nat Struct Biol, 7 Suppl: 973-977, 2000.
86. Dobson, C. M. Chemical space and biology. Nature, 432: 824-828, 2004.
87. Fry, D. C. and Vassilev, L. T. Targeting protein-protein interactions for cancer therapy. J Mol Med, 2005.
88. Hajduk, P. J., Huth, J. R., and Fesik, S. W. Druggability indices for protein targets derived from NMR-based screening data. J Med Chem, 48: 2518-2525, 2005.
89. Keskin, O., Ma, B., Rogale, K., Gunasekaran, K., and Nussinov, R. Protein-protein interactions: organization, cooperativity and mapping in a bottom-up Systems Biology approach. Phys Biol, 2: S24S35, 2005.
90. Keskin, O., Ma, B., and Nussinov, R. Hot regions in protein—protein interactions: the organization and contribution of structurally conserved hot spot residues. J Mol Biol, 345: 1281-1294, 2005.
91. Pellecchia, M., Meininger, D., Dong, Q., Chang, E., Jack, R., and Sem, D. S, NMR-based structural characterization of large protein-ligand interactions. J Biomol NMR, 22: 165-173., 2002.
92. Palmer, A. G., 3rd NMR characterization of the dynamics of biomacromolecules. Chem Rev, 104: 36233640, 2004.
93. Riek, R., Wider, G., Pervushin, K., and Wuthrich, K. Polarization transfer by cross-correlated relaxation in solution NMR with very large molecules. Proc Natl Acad Sci U S A, 96: 4918-4923., 1999.
94. Pervushin, K., Braun, D., Fernandez, C., and Wuthrich, K. [15N, 1H]/[13C, 1H]-TROSY for simultaneous detection of backbone 15N-1H, aromatic 13C-1H and side-chain 15N-1H2 correlations in large proteins. J Biomol NMR, 17: 195-202., 2000.
95. Pellecchia, M., Becattini, B., Crowell, K. J., Fattorusso, R., Forino, M., Fragai, M., Jung, D., Mustelin, T., and Tautz, L. NMR-based techniques in the hit identification and optimisation processes. Expert Opin Ther Targets, 8: 597-611, 2004.
96. Pellecchia, M., Sem, D. S., and Wuthrich, K. NMR in drug discovery. Nat Rev Drug Discov, 1: 211-219., 2002.
97. Shuker, S. B., Hajduk, P. J., Meadows, R. P., and Fesik, S. W. Discovering high-affinity ligands for proteins: SAR by NMR. Science, 274: 1531-1534., 1996.
98. Schade, M. and Oschkinat, H. NMR fragment screening: tackling protein-protein interaction targets. Curr Opin Drug Discov Devel, 8: 365-373, 2005. 18
99. Zartler, E. R., Hanson, J., Jones, B. E., Kline, A. D., Martin, G., Mo, H., Shapiro, M. J., Wang, R., Wu, H., and Yan, J. RAMPED-UP NMR: multiplexed NMR-based screening for drug discovery. J Am Chem Soc, 125: 10941-10946, 2003.
100. Zartler, E. R. and Shapiro, M. J. Fragonomics: fragment-based drug discovery. Curr Opin Chem Biol, 9: 366-370, 2005.
101. Pellecchia, M. Solution nuclear magnetic resonance spectroscopy techniques for probing intermolecular interactions. Chem Biol, 12: 961-971, 2005.
102. Roehrl, M. H., Kang, S., Aramburu, J., Wagner, G., Rao, A., and Hogan, P. G. Selective inhibition of calcineurin-NFAT signaling by blocking protein-protein interaction with small organic molecules. Proc Natl Acad Sci USA, 101: 7554-7559, 2004.
103. Aramburu, J., Yaffe, M. B., Lopez-Rodriguez, C., Cantley, L. C., Hogan, P. G., and Rao, A. Affinity-driven peptide selection of an NFAT inhibitor more selective than cyclosporin A. Science, 285: 21292133, 1999.
104. May, M. J., D′Acquisto, F., Madge, L. A., Glockner, J., Pober, J. S., and Ghosh, S. Selective inhibition of NF-kappaB activation by a peptide that blocks the interaction of NEMO with the IkappaB kinase complex. Science, 289: 1550-1554, 2000.
105. Polo, J. M., Dell'Oso, T., Ranuncolo, S. M., Cerchietti, L., Beck, D., Da Silva, G. F., Prive, G. G., Licht, J. D., and Melnick, A. Specific peptide interference reveals BCL6 transcriptional and oncogenic mechanisms in B-cell lymphoma cells. Nat Med, 10: 1329-1335, 2004.
106. Dzau, V. J. and Lopez-Ilasaca, M. Searching for transcriptional regulators of Ang II-induced vascular pathology. J Clin Invest, 115: 2319-2322, 2005.
107. Lipinski, C. A. Drug-like properties and the causes of poor solubility and poor permeability. J Pharmacol Toxicol Methods, 44: 235-249., 2000.
108. Lipinski, C. A., Lombardo, F., Dominy, B. W., and Feeney, P. J. Experimental and computational approaches to estimate solubility and permeability in drug discovery and development settings. Adv Drug Deliv Rev, 46: 3-26, 2001.
109. Lipinski, C. and Hopkins, A. Navigating chemical space for biology and medicine. Nature, 432: 855-861, 2004.
110. Eldridge, M. D., Murray, C. W., Auton, T. R., Paolini, G. V., and Mee, R. P. Empirical scoring functions: I. The development of a fast empirical scoring function to estimate the binding affinity of ligands in receptor complexes. J Comput Aided Mol Des, 11: 425-445, 1997.
111. Shoichet, B. K. Virtual screening of chemical libraries. Nature, 432: 862-865, 2004.
112. Bajorath, J. Predicting novel proteins and their interactions. Drug Discov Today, 7: 945-946, 2002.
113. Bajorath, J. Integration of virtual and high-throughput screening. Nat Rev Drug Discov, 1: 882-894, 2002.
114. Hindle, S. A., Rarey, M., Buning, C., and Lengaue, T. Flexible docking under pharmacophore type constraints. J Comput Aided Mol Des, 16: 129-149, 2002.
115. Ripka, A. S, and Rich, D. H. Peptidomimetic design. Curr Opin Chem Biol, 2: 441-452., 1998.
116. Ripka, A. S., Satyshur, K. A., Bohacek, R. S., and Rich, D. H. Aspartic protease inhibitors designed from computer-generated templates bind as predicted. Org Lett, 3: 2309-2312., 2001.
117. Greenidge, P. A., Carlsson, B., Bladh, L. G., and Gillner, M. Pharmacophores incorporating numerous excluded volumes defined by X-ray crystallographic structure in three-dimensional database searching: application to the thyroid hormone receptor. J Med Chem, 41: 2503-2512., 1998.
118. Pajeva, I. K. and Wiese, M. Pharmacophore model of drugs involved in P-glycoprotein multidrug resistance: explanation of structural variety (hypothesis). J Med Chem, 45: 5671-5686., 2002.
119. Patel, Y., Gillet, V. J., Bravi, G., and Leach, A. R. A comparison of the pharmacophore identification programs: Catalyst, DISCO and GASP. J Comput Aided Mol Des, 16: 653-681., 2002.
120. Greenidge, P. A., Merette, S. A., Beck, R., Dodson, G., Goodwin, C. A., Scully, M. F., Spencer, J., Weiser, J., and Deadman, J. J. Generation of ligand conformations in continuum solvent consistent with protein active site topology: application to thrombin. J Med Chem, 46: 1293-1305., 2003.
121. Pufall, M. A., Lee, G. M., Nelson, M. L., Kang, H. S., Velyvis, A., Kay, L. E., McIntosh, L. P., and Graves, B. J. Variable control of Ets-1 DNA binding by multiple phosphates in an unstructured region. Science, 309: 142-145, 2005.
122. Rarey, M., Kramer, B., Lengauer, T., and Klebe, G. A fast flexible docking method using an incremental construction algorithm. J Mol Biol, 261: 470-489, 1996.
123. Bohm, H. J. LUDI: rule-based automatic design of new substituents for enzyme inhibitor leads. J Comput Aided Mol Des, 6: 593-606, 1992. 20
124. Klebe, G. The use of composite crystal-field environments in molecular recognition and the de novo design of protein ligands. J Mol Biol, 237: 212-235, 1994.
125. Kramer, B., Rarey, M., and Lengauer, T. Evaluation of the FLEXX incremental construction algorithm for protein-ligand docking. Proteins, 37: 228-241, 1999.
126. Bleicher, K. H., Bohm, H. J., Muller, K., and Alanine, A. I. Hit and lead generation: beyond high-throughput screening. Nat Rev Drug Discov, 2: 369-378, 2003.
127. Boyce, M., Bryant, K. F., Jousse, C., Long, K., Harding, H. P., Scheuner, D., Kaufman, R. J., Ma, D., Coen, D. M., Ron, D., and Yuan, J. A selective inhibitor of eIF2alpha dephosphorylation protects cells from ER stress. Science, 307: 935-939, 2005.
128. Gurova, K. V., Hill, J. E., Guo, C., Prokvolit, A., Burdelya, L. G., Samoylova, E., Khodyakova, A. V., Ganaphthi, R., Ganaphthi, M., Tararova, N. D., Bosykh, D., Lvovskiy, D., Webb, T. R., Stark, G. R., and Gudkov, A. V. Small molecules that reactivate p53 in renal cell carcinoma reveal a NF-kappaBdependent mechanism of p53 suppression in tumors. Proc Natl Acad Sci USA, 102: 17448-17453, 2005.
129. Hung, D. T., Shakhnovich, E. A., Pierson, E., and Mekalanos, J. J. Small-molecule inhibitor of Vibrio cholerae virulence and intestinal colonization. Science, 310: 670-674, 2005.
130. Gurova, K. V., Hill, J. E., Razorenova, O. V., Chumakov, P. M., and Gudkov, A. V. p53 pathway in renal cell carcinoma is repressed by a dominant mechanism. Cancer Res, 64: 1951-1958, 2004.
131. Godden, J. W., Stahura, F. L., and Bajorath, J. Anatomy of fingerprint search calculations on structurally diverse sets of active compounds. J Chem Inf Model, 45: 1812-1819, 2005.
132. Holliday, J. D., Hu, C. Y., and Willett, P. Grouping of coefficients for the calculation of inter-molecular similarity and dissimilarity using 2D fragment bit-strings. Comb Chem High Throughput Screen, 5: 155166, 2002.
133. Xue, L., Godden, J. W., Stahura, F. L., and Bajorath, J. Design and evaluation of a molecular fingerprint involving the transformation of property descriptor values into a binary classification scheme. J Chem Inf Comput Sci, 43: 1151-1157, 2003.
134. Xue, L., Godden, J. W., and Bajorath, J. Mini-fingerprints for virtual screening: design principles and generation of novel prototypes based on information theory. SAR QSAR Environ Res, 14: 27-40, 2003.
135. Xue, L., Godden, J. W., Stahura, F. L., and Bajorath, J. Profile scaling increases the similarity search performance of molecular fingerprints containing numerical descriptors and structural keys. J Chem Inf Comput Sci, 43: 1218-1225, 2003.
136. Godden, J. W., Xue, L., Stahura, F. L., and Bajorath, J. Searching for molecules with similar biological activity: analysis by fingerprint profiling. Pac Symp Biocomput 566-575, 2000.
137. Xue, L., Stahura, F. L., Godden, J. W., and Bajorath, J. Fingerprint scaling increases the probability of identifying molecules with similar activity in virtual screening calculations. J Chem Inf Comput Sci, 41: 746-753, 2001.
138. Godden, J. W., Furr, J. R., Xue, L., Stahura, F. L., and Bajorath, J. Molecular similarity analysis and virtual screening by mapping of consensus positions in binary-transformed chemical descriptor spaces with variable dimensionality. J Chem Inf Comput Sci, 44: 21-29, 2004.
139. Kontoyianni, M., McClellan, L. M., and Sokol, G. S. Evaluation of docking performance: comparative data on docking algorithms. J Med Chem, 47: 558-565, 2004.
140. Kontoyianni, M., Sokol, G. S., and McClellan, L. M. Evaluation of library ranking efficacy in virtual screening. J Comput Chem, 26: 11-22, 2005.
141. Morelli, X. a. R., A. C. Acceleration of the Drug Discovery Process: A Combinatorial Approach Using NMR Spectroscopy and Virtual Screening. Current Computer-Aided Drug Design, In Revision, 2006.
142. Dube, A., Akbarali, Y., Sato, T. N., Libermann, T. A., and Oettgen, P. Role of the Ets transcription factors in the regulation of the vascular-specific Tie2 gene. Circ Res, 84: 1177-1185, 1999.
143. Carr, R. A., Congreve, M., Murray, C. W., and Rees, D. C. Fragment-based lead discovery: leads by design. Drug Discov Today, 10: 987-992, 2005.
144. Rees, D. C., Congreve, M., Murray, C. W., and Carr, R. Fragment-based lead discovery. Nat Rev Drug Discov, 3: 660-672, 2004.
145. Boger, D. L., Fink, B. E., Brunette, S. R., Tse, W. C., and Hedrick, M. P. A simple, high-resolution method for establishing DNA binding affinity and sequence selectivity. J Am Chem Soc, 123: 58785891, 2001.
146. Iljin, K., Dube, A., Kontusaari, S., Korhonen, J., Lahtinen, I., Oettgen, P., and Alitalo, K. Role of ets factors in the activity and endothelial cell specificity of the mouse Tie gene promoter. Faseb J, 13: 377386, 1999. 21
147. Zhan, Y., Kim, S., Izumi, Y., Izumiya, Y., Nakao, T., Miyazaki, H., and Iwao, H. Role of JNK, p38, and ERK in platelet-derived growth factor-induced vascular proliferation, migration, and gene expression. Arterioscler Thromb Vasc Biol, 23: 795-801, 2003.
148. Oettgen, P., Akbarali, Y., Boltax, J., Best, J., Kunsch, C., and Libermann, T. A. Characterization of NERF, a novel transcription factor related to the Ets factor ELF-1. Mol Cell Biol, 16: 5091-5106, 1996.
149. Wells, J., Graveel, C. R., Bartley, S. M., Madore, S. J., and Farnham, P. J. The identification of E2F1 specific target genes. Proc Natl Acad Sci USA, 99: 3890-3895, 2002.
150. Weinmann, A. S., Yan, P. S., Oberley, M. J., Huang, T. H., and Farnham, P. J. Isolating human transcription factor targets by coupling chromatin immunoprecipitation and CpG island microarray analysis. Genes Dev, 16: 235-244, 2002.
151. Boyd, K. E. and Farnham, P. J. Myc versus USF: discrimination at the cad gene is determined by core promoter elements. Mol Cell Biol, 17: 2529-2537, 1997.
152. Goldsmith-Fischman, S, and Honig, B. Structural genomics: computational methods for structure analysis. Protein Sci, 12: 1813-1821, 2003.
153. Werner, M. H., Clore, M., Fisher, C. L., Fisher, R. J., Trinh, L., Shiloach, J., and Gronenborn, A. M. The solution structure of the human ETS1-DNA complex reveals a novel mode of binding and true side chain intercalation. Cell, 83: 761-771, 1995.
154. Werner, M. H., Clore, G. M., Gronenborn, A. M., Kondoh, A., and Fisher, R. J. Refolding proteins by gel filtration chromatography. FEBS Lett, 345: 125-130, 1994.
155. Houbaviy, H. B. and Burley, S. K. Thermodynamic analysis of the interaction between YY1 and the AAV P5 promoter initiator element. Chem Biol, 8: 179-187, 2001.
156. Hajduk, P. J., Betz, S. F., Mack, J., Ruan, X., Towne, D. L., Lerner, C. G., Beutel, B. A., and Fesik, S. W. A strategy for high-throughput assay development using leads derived from nuclear magnetic resonance-based screening. J Biomol Screen, 7: 429-432, 2002.
157. Clarkson, J. and Campbell, I. D. Studies of protein-ligand interactions by NMR. Biochem Soc Trans, 31: 1006-1009, 2003.
158. Bagby, S., Tong, K. I., and Ikura, M. Optimization of protein solubility and stability for protein nuclear magnetic resonance. Methods Enzymol, 339: 20-41, 2001.
159. Zimmerman, D. E., Kulikowski, C. A., Huang, Y., Feng, W., Tashiro, M., Shimotakahara, S., Chien, C., Powers, R., and Montelione, G. T. Automated analysis of protein NMR assignments using methods from artificial intelligence. J Mol Biol, 269: 592-610, 1997.
160. Linge, J. P., Habeck, M., Rieping, W., and Nilges, M. ARIA: automated NOE assignment and NMR structure calculation. Bioinformatics, 19: 315-316, 2003.
161. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. Crystallography & NMR system: A new software suite for macromolecular structure determination. Acta Crystallogr D Biol Crystallogr, 54 (Pt 5): 905-921, 1998.

The invention has been described in detail in particular references to the preferred embodiments thereof. However, it will be appreciated that modifications and improvements within the spirit and scope of this invention may be made by those skilled in the art upon considering the present disclosure.

1. A method of identifying a small molecule candidate agent capable of modulating transcription factor function such that the function/expression of a target transcription factor and/or proteins that are downstream of this target protein comprising the steps of:
a) screening small molecule libraries using in silico high throughput docking for candidate small molecules/agents that are selectively identified for their ability to target and disrupt the transcription factor-DNA interface through unique transcription factor and/or DNA descriptors that are defined within a pharmacophore; and
b) testing/evaluating the candidate agents identified in step (a) through one or more in vitro assays for their ability to modulate transcription factor function including expression of this target protein and/or proteins that are downstream of the target transcription factor.

2. The method of claim 1 wherein further the candidate agents comprised of unique chemical scaffolds as identified in step (b) are optimized for their ability to modulate the function and/or expression of the transcription factor target protein and/or proteins downstream of this transcription factor comprising testing the candidate agents in assays selected from the group consisting of: 1) in silico quantitative structure activity relationships; 2) similarity fingerprint searching; and 3) NMR spectroscopy driven structural chemistry wherein optimization results in greater modulation of protein expression.

3. The method of claim 1 wherein the candidate agent modulation of protein expression is down-regulated.

4. The method of claim 1 wherein the candidate agent modulation of protein expression is up-regulated.

5. The method of claim 1 wherein the Transcription Factor is a member of the ETS family of transcription factors.

6. The method of claim 5 wherein the member of the ETS family of transcription factors is Ets-1.

7. The method of claim 1 wherein the small molecule library is selected from the group consisting of: 1) the National Cancer Institute's Diversity Set; 2) the National Cancer Institute's Open Chemical Repository; 3) the Chembridge Library DIVERSet; 4) the Maybridge Library; 5) the Platinum Collection from Asinex; and the Zinc Database including the Natural Product Library.

8. The method of claim 1 wherein the pharmacophore descriptors included for the transcription factor protein comprise of hydrogen bond acceptors, hydrogen bond donors, hydrophobic disposition and the geometry of the protein's molecular scaffold (DBD backbone) and critical amino acids within the DNA binding domain of the transcription factor target.

9. The method of claim 1 wherein the pharmacophore definitions for the protein are computationally determined using a genetic algorithm and have been demonstrated to be critical through mutagenesis data, DNA binding data and/or other in vitro assays as identified within the supporting documentation.

10. A method of preventing or treating a patient with an ETS-mediated disease/disorder or susceptibility to an ETS-mediated disease/disorder comprising the administration to the patient in need of such treatment or prevention a therapeutically effective amount of a compound identified by a method of claim 1.

11. A method of preventing or treating a patient with a disease/disorder or susceptibility to a disease/disorder involving inflammation or angiogenesis, or a patient suffering from cancer, comprising the administration to the patient in need of such treatment or prevention a therapeutically effective amount of a compound identified by a method of claim 1.

12. The method of claim 11 wherein the disease/disorder is rheumatoid arthritis; an inflammatory bowel disease; cancer; prostate, breast, colon, ovarian, lung or stomach cancer; artherosclerosis; bacterial sepsis; hypertension; or restenosis.

13-19. (canceled)

20. The method of claim 11 wherein further the compound or salt thereof interacts with and abrogates the function of the DNA Binding Domain of a Transcription Factor protein involved in inflammation or angiogenesis.

21. The method of claim 20 wherein the interaction of the compound or salt thereof with the DNA Binding Domain of a Transcription Factor Protein involved in inflammation or angiogenesis results in the inhibition of the target proteins function/expression of in addition to those proteins that are downstream of this transcription factor and are involved in inflammation or angiogenesis.

22. A method of claim 10 wherein the transcription factor is selected from the group consisting of ESE-1, Ets-1, Ets-2, SAP, PDEF, ERG, ETV-1, ELK-1, Erp-1, TEL-1, TEL-2, PU.1 and Fli-1, NRF-2 and ELF-1.

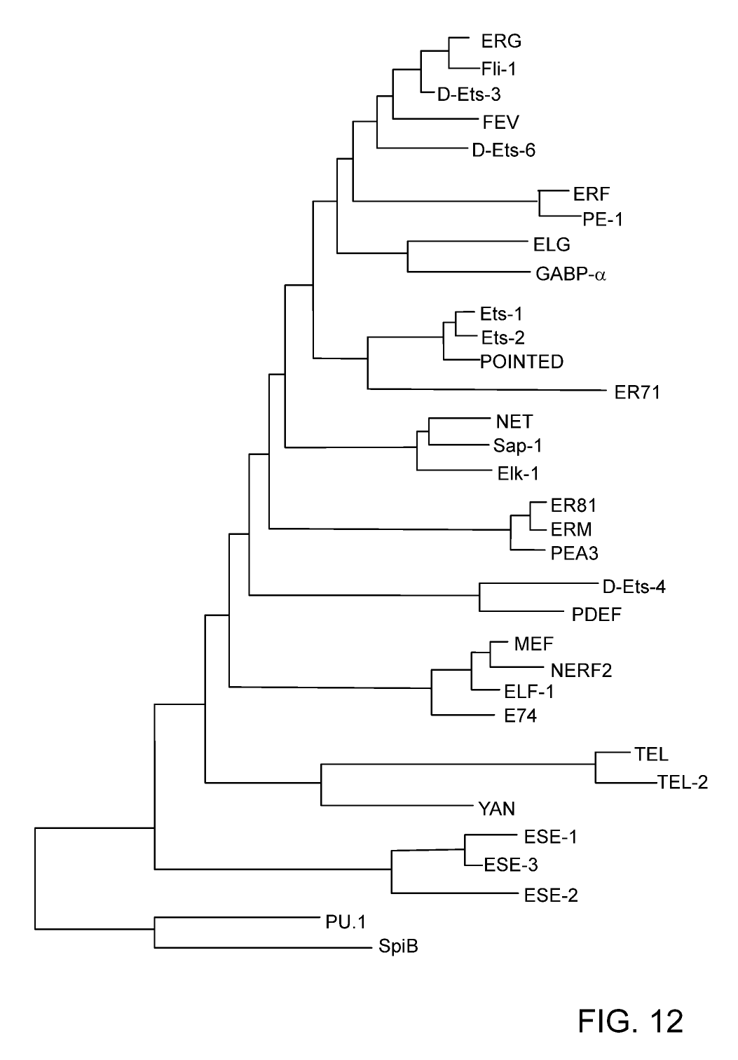

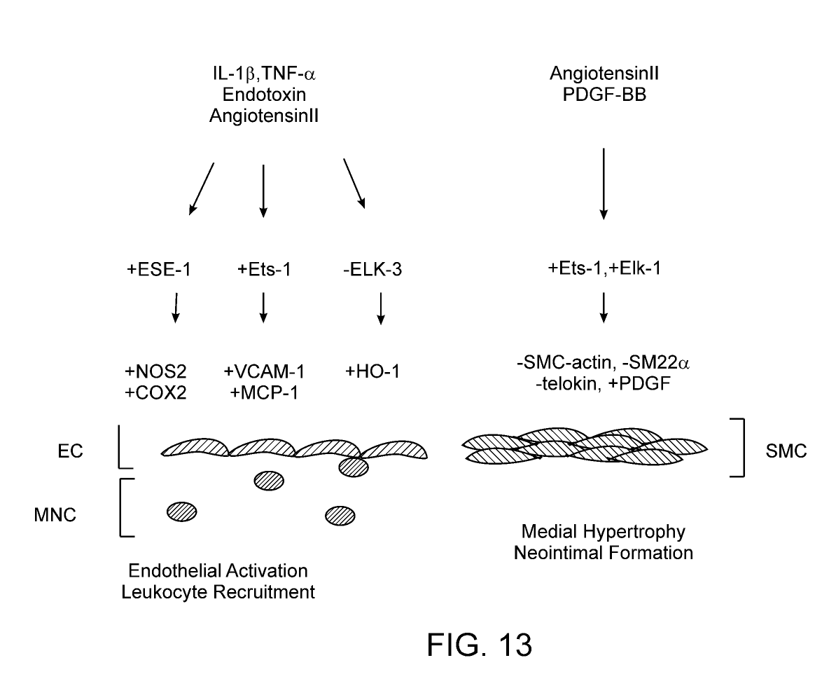

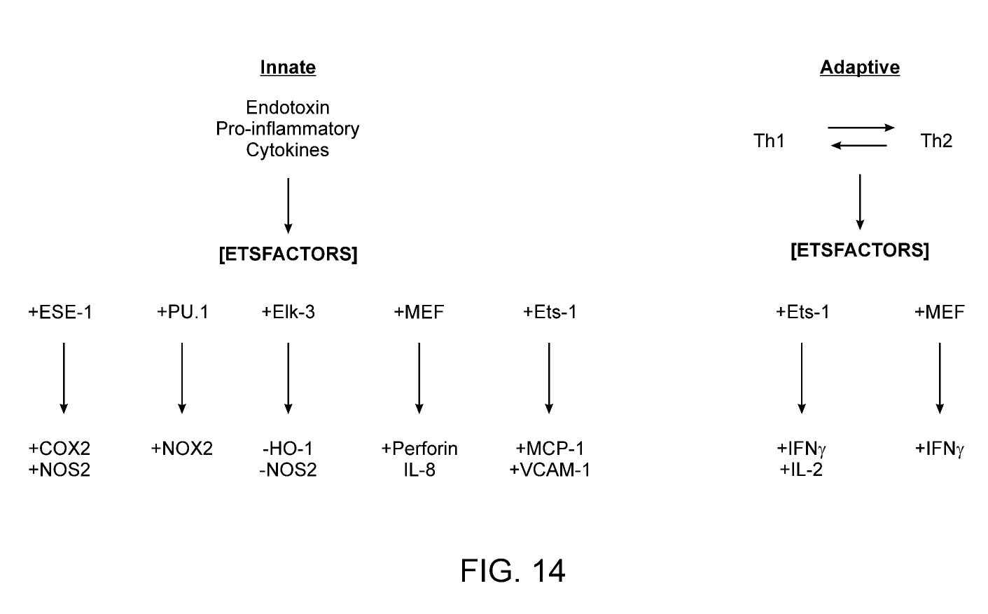

23. A method of claim 10 wherein the transcription factor is selected from the group consisting of any of the transcription factors listed in FIG. 12.

24. The method of claim 10 wherein the compound or salt thereof is as NCI 371776, 2-[1-(4-Ethoxy-phenyl)-2-nitro-ethylsulfanyl]-phenylamine).

25. The method of claim 10 wherein the compound or salt thereof is NCI 371777, 2-{[1-(1,3-benzodioxol-5-yl)-2-nitroethyl]thio}aniline.

26. A method of inhibiting angiogenesis in a patient comprising the administration to the patient in need of such angiogenesis inhibition a therapeutically effective amount of a compound identified by a method of claim 1.

27. A method of treating or preventing a condition selected from the group consisting of Inflammatory Bowel Disease, Rheumatoid Arthritis, Psoriasis, and Diabetic Retinopathy in a patient in need thereof, the method comprising the administration to the patient a therapeutically effective amount of a compound identified by a method of claim 1.

28. A compound which is a small molecule candidate agent identified by the method of claim 1, or a pharmaceutically acceptable salt thereof.

29-30. (canceled)

31. A compound represented by the formula A-D, in which A is an aromatic moiety or other molecular fragment capable of interacting with a DNA Binding Domain of a Transcription Factor involved in inflammation or angiogenesis; and D is a moiety capable of interacting with a nucleic acid to which the transcription factor binds; and pharmaceutically acceptable esters, salts, and prodrugs thereof, wherein the compound is capable of modulating the function and/or expression of the transcription factor target and subsequent downstream proteins.

32. A pharmaceutical composition comprising a compound of claim 31, or a pharmaceutically acceptable salt thereof, together with a pharmaceutically acceptable carrier.

33. A pharmaceutical composition comprising a compound of claim 1, or a pharmaceutically acceptable salt thereof, together with a pharmaceutically acceptable carrier.

34. A kit for treating an inflammation or angiogenesis in a subject, the kit comprising a compound of claim 1, pharmaceutically acceptable esters, salts, and prodrugs thereof, and instructions for use.

35-40. (canceled)

41. The compound of claim 31, wherein the compound is selected from the group consisting of:

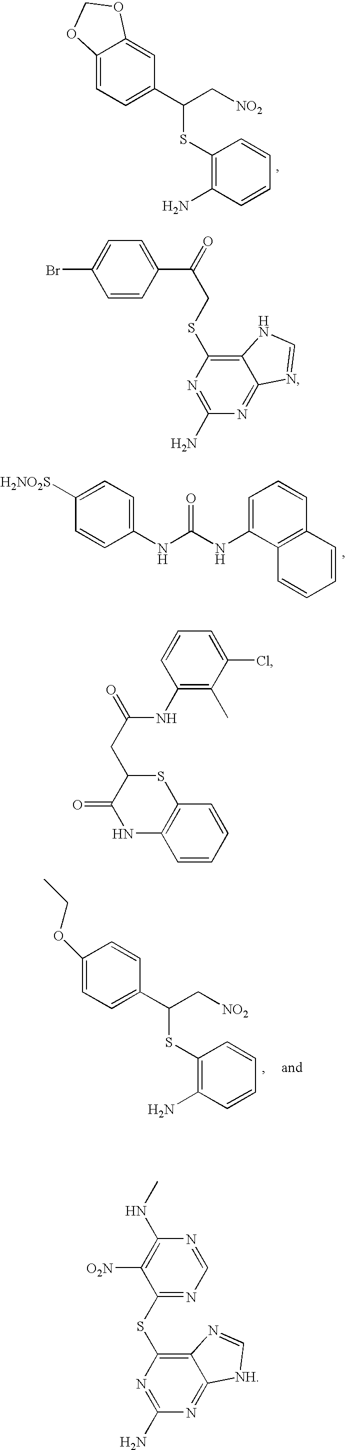

42-54. (canceled)

55. A method of identifying a small molecule candidate agent capable of modulating transcription factor function such that the function/expression of a target transcription factor and/or proteins that are downstream of this target protein comprising the steps of:
a) screening small molecule libraries using in silico high throughput docking for candidate small molecules/agents that are selectively identified for their ability to target and disrupt the transcription factor-DNA interaction interface through unique transcription factor DNA binding domain descriptors that are defined within the target (transcription factor DNA binding domain hot sports within the pharmacophore; and
b) testing/evaluating the candidate agents identified in step (a) through one or more in vitro assays for their ability to modulate transcription factor function including expression of this target protein and/or proteins that are downstream of the target transcription factor.
